(19) United States
(12) Patent Application Publication
(10) Pub. No.: US 2024/0374779 A1
(43) Pub. Date: Nov. 14, 2024

(54) ANTIMICROBIAL ARTICLES, METHODS OF MAKING AND USING SAME

(71) Applicant: TERASAKI INSTITUTE FOR BIOMEDICAL INNOVATION, Los Angeles, CA (US)

(72) Inventors: Seyedvahid Hosseini, Los Angeles, CA (US); Alireza Khademhosseini, Los Angeles, CA (US)

(21) Appl. No.: 18/286,309

(22) PCT Filed: Mar. 30, 2022
(86) PCT No.: PCT/US22/22449

§ 371 Date: Oct. 10, 2023

Related U.S. Application Data
(60) Provisional application No. 63/175,240, filed on Apr. 15, 2021.

(51) Int. Cl. A61L 15/26; A61L 15/18; A61L 15/44; D06M 11/50; D06M 15/507

(52) U.S. Cl. CPC A61L 15/26; A61L 15/18; A61L 15/44; D06M 11/50; D06M 15/507; A61L 2300/11

(57) ABSTRACT

Provided are articles having a substrate and a polymer matrix. The polymer matrix includes a hydrogen peroxide releasing compound dispersed within the polymer matrix. The polymer matrix is provided on a surface of the substrate. Also provided are methods of forming the articles of the disclosure and kits for preparing the articles of the disclosure.

### FIELD OF THE DISCLOSURE

The disclosure generally relates to articles that can have antimicrobial properties. The articles include a substrate and a polymer matrix. The polymer matrix can be in contact with the substrate, e.g., a surface of the substrate. The polymer matrix includes a hydrogen peroxide releasing compound. The hydrogen peroxide releasing compound is dispersed within the polymer matrix. Also provided are methods of forming the articles of the disclosure.

### BRIEF DESCRIPTION OF RELATED TECHNOLOGY

Various types of antimicrobial coatings and surface treatments are known. For example, cationic polymer grafted surfaces have been shown to have antibacterial and antiviral effects. However, limitations of commercial antibacterial and antiviral coatings include the relative incapability of releasing the active agents to the surrounding environment, and the requirement of direct contact in order for inactivation to occur. Thus, the efficiency of these commercial coatings can be limited if large amounts of viruses, bacteria or organic contaminant; such as dust, interfering substances, or dead virus and bacterial particles cover the coating, thereby inhibiting inactivation by the coating. As a result, the antimicrobial or antiviral activity of these coatings declines during extended use and, particularly, in contaminated areas. Additionally, commercial filters, such as face masks, HEPA filters, AC filters, etc., are generally able to trap contaminants, but do not inactivate any bacteria, viruses, or the like that become trapped therein. Thus, when these filters are changed or disposed of, the microbial contaminants trapped therein can be again released into the environment, creating health risks to the consumer.

Accordingly, improved articles are needed in order to trap and/or inactivate microbes, such as bacteria, fungi, and viruses.

### SUMMARY

Provided herein are articles comprising a substrate, and a polymer matrix comprising a hydrogen peroxide releasing compound dispersed within the polymer matrix, wherein the polymer matrix is in contact with the substrate.

In embodiments, the substrate comprises a nonwoven material. In embodiments, the substrate comprises a woven material, a polymeric material, or a metal alloy. In embodiments, the nonwoven material or the woven material comprises polypropylene fiber, polyacrylic acid fiber, polyurethane fiber, polyester fiber, fiberglass fiber, cellulose, hemp, jute, flax, ramie, sisal, bagasse, banana fiber, lacebark, silk, sinew, catgut, wool, sea silk, mohair, angora, cashmere, collagen, actin, nylon, dacron, rayon, bamboo fiber, modal, diacetate fiber, triacetate fiber, copolyester, viscose, polylactide, polyethylene terephthalate, or a combination thereof. In embodiments, the polymeric material comprises high-density polyethylene (HDPE) or polyethylene terephthalate (PET). In embodiments, the metal alloy comprises steel.

In embodiments, the hydrogen peroxide releasing compound comprises one or more of urea hydrogen peroxide (UHP) and a metal peroxide. In embodiments, the metal peroxide comprises one or more of calcium peroxide, magnesium peroxide, silver peroxide, copper peroxide, and zinc peroxide.

In embodiments, the polymer matrix comprises a biodegradable polymer selected from one or more of polyglycolic acid (PGA), polycaprolactone (PCL), polylactic acid (PLA), polyurethane (PU), a polyester, and poly (lactic-co-glycolic acid) (PLGA). In embodiments, the polymer matrix comprises a nonbiodegradable polymer.

In embodiments, the hydrogen peroxide releasing compound is present in an amount in a range of about 5 wt % to about 95 wt %, based on the total weight of the polymer matrix. In embodiments, the polymer is present in an amount in a range of 5 wt % to about 95 wt %, based on the total weight of the polymer matrix. In embodiments, the hydrogen peroxide releasing compound and the polymer are present in a weight ratio in a range of about 1:19 to about 19:1. In embodiments, the weight ratio is in a range of about 1:10 to about 15:1.

In embodiments, the polymer matrix is provided as a coating, a particle, or both.

In embodiments, the article is an antimicrobial article effective against one or more of a fungus, gram-positive bacteria, gram-negative bacteria, spore of bacteria, and a virus. In embodiments, the fungus comprises one or more of Candida albicans, Candida auris, Cryptococus gattii, Cryptococcus neoformans, Blastomyces dermatitidis, Paracoccidioides brasiliensis, Paracoccidioides lutzii, Coccidioides immitis, Coccidioides posadasii, Histoplasma capsulatum, Fusarium solani, Fusarium oxysporum, Fusarium verticillidioidis, Fusarium monoliforme, Aspergillus niger, Aspergillus flava, Aspergillus fumigatus, Aspergillis parasiticus, and Pneumocystis jirovecii. In embodiments, the gram-negative bacteria comprise one or more of Escherichia coli, Pseudomonas aeruginosa, Klebsiella pneumoniae, Klebsiella oxytoca, Klebsiella terrigena, Klebsiella planticola, Acinetobacter baumannii, Acinetobacter calcoaceticus, and Mycobacterium tuberculosis. In embodiments, the gram-positive bacteria comprises Staphylococcus aureus, methicillin-resistant Staphylococcus aureus, Clostridium difficile, and vancomycin-resistant Enterococcus. In embodiments, the virus causes a viral disease selected from one or more of COVID-19, chickenpox, influenza, herpes, human immunodeficiency virus (HIV/AIDS), human papillomavirus (HPV), infectious mononucleosis, mumps, measles, rubella, shingles, viral gastroenteritis, viral hepatitis, viral meningitis, and viral pneumonia.

In embodiments, the article is bacteriostatic. In embodiments, the article is bactericidal.

In embodiments, hydrogen peroxide is released from the hydrogen peroxide releasing compound when the article is exposed to a relative humidity (RH) of at least about 5%. In embodiments, hydrogen peroxide is released from the hydrogen peroxide releasing compound when the article is exposed to a relative humidity (RH) of about 5% to about 25%. In embodiments, hydrogen peroxide is released from the hydrogen peroxide releasing compound when the article is exposed to a relative humidity (RH) of at least about 30%. In embodiments, hydrogen peroxide is released from the hydrogen peroxide releasing compound when the article is exposed to a relative humidity (RH) of about 90% to about 100%.

In embodiments, the article is incorporated into a filter, a lining material, a medical device, a tool, or a bedding material. In embodiments, the bedding material is a mattress, a mattress pad, a sheet, a pillow case, a comforter, or a duvet cover. In embodiments, the filter is a face mask, a respirator, an HVAC filter, a vacuum filter, or a HEPA filter. In embodiments, the medical device is a disinfectant wipe, a wound dressing, or a biohazard reservoir. In embodiments, the lining material is provided in an interior of a bag. In embodiments, the tool is a mobile phone, a cosmetic tool, a utensil, or a minor surgical tool.

In embodiments, the hydrogen peroxide releasing compound comprises UHP and the polymer matrix comprises PCL. In embodiments, the UHP is present in an amount of about 92 wt %, based on the total weight of the polymer matrix, and the PCL is present in an amount of about 8 wt %, based on the total weight of the polymer matrix. In embodiments, the UHP is present in an amount of about 86 wt %, based on the total weight of the polymer matrix, and the PCL is present in an amount of about 14 wt %, based on the total weight of the polymer matrix. In embodiments, the UHP is present in an amount of about 60 wt %, based on the total weight of the polymer matrix, and the PCL is present in an amount of about 40 wt %, based on the total weight of the polymer matrix. In embodiments, the UHP is present in an amount of about 50 wt %, based on the total weight of the polymer matrix, and the PCL is present in an amount of about 50 wt %, based on the total weight of the polymer matrix.

In embodiments, the hydrogen peroxide releasing compound comprises UHP and the polymer matrix comprises PLGA. In embodiments, the UHP is present in an amount of about 40 wt %, based on the total weight of the polymer matrix, and the PLGA is present in an amount of about 60 wt %, based on the total weight of the polymer matrix. In embodiments, the UHP is present in an amount of about 20 wt %, based on the total weight of the polymer matrix, and the PLGA is present in an amount of about 80 wt %, based on the total weight of the polymer matrix. In embodiments, the UHP is present in an amount of about 10 wt %, based on the total weight of the polymer matrix, and the PLGA is present in an amount of about 90 wt %, based on the total weight of the polymer matrix.

In embodiments, the article is permeable to a gas.

Also provided are methods of forming an article, comprising contacting a surface of a substrate with a mixture comprising a hydrogen peroxide releasing compound and a polymer to provide the article.

In embodiments, contacting comprises dipping, brushing, melt-spinning, extruding, molding, or spraying.

In embodiments, the mixture further comprises a solvent for the polymer, the hydrogen peroxide releasing compound, or both. In embodiments, the mixture is a solution, wherein each of the polymer and the hydrogen peroxide releasing compound are dissolved in the solvent. In embodiments, the polymer is dissolved in the solvent to provide a polymeric solution, and the hydrogen peroxide releasing compound is dispersed in the polymeric solution. In embodiments, the solvent comprises an organic solvent. In embodiments, the organic solvent comprises one or more of dichloromethane, acetonitrile, ethanol, methanol, butanol, propanol, and acetone.

In embodiments, the mixture is free of one or more of water, ferric ion, and a peroxidase.

In embodiments, the hydrogen peroxide releasing compound is present in the mixture in an amount in a range of about 5 wt % to about 95 wt %, based on the total weight of the hydrogen peroxide releasing compound and the polymer. In embodiments, the hydrogen peroxide releasing compound is present in the mixture in an amount in a range of about 1% (w/v) to about 20% (w/v).

In embodiments, the polymer is present in the mixture in an amount in a range of about 5 wt % to about 95 wt %, based on the total weight of the hydrogen peroxide releasing compound and the polymer. In embodiments, the polymer is present in the mixture in an amount in a range of about 1% (w/v) to about 10% (w/v).

In embodiments, the hydrogen peroxide releasing compound and the polymer are present in a weight ratio of about 1:10 to about 15:1.

In embodiments, the hydrogen peroxide releasing compound comprises UHP, the polymer comprises PCL, and the solvent comprise dichloromethane. In embodiments, the UHP is present in the mixture in an amount of about 12% (w/v), and the PCL is present in the mixture in an amount of about 1% (w/v). In embodiments, the UHP is present in the mixture in an amount of about 6% (w/v), and the PCL is present in the mixture in an amount of about 1% (w/v). In embodiments, the UHP is present in the mixture in an amount of about 1% (w/v), and the PCL is present in the mixture in an amount of about 1% (w/v).

In embodiments, the hydrogen peroxide releasing compound comprises UHP, the polymer comprises PLGA, and the solvent comprise acetonitrile. In embodiments, the UHP is present in the mixture in an amount of about 6% (w/v), and the PLGA is present in the mixture in an amount of about 4% (w/v). In embodiments, the UHP is present in the mixture in an amount of about 5% (w/v), and the PLGA is present in the mixture in an amount of about 5% (w/v). In embodiments, the UHP is present in the mixture in an amount of about 4% (w/v), and the PLGA is present in the mixture in an amount of about 6% (w/v). In embodiments, the UHP is present in the mixture in an amount of about 2% (w/v), and the PLGA is present in the mixture in an amount of about 8% (w/v). In embodiments, the UHP is present in the mixture in an amount of about 1% (w/v), and the PLGA is present in the mixture in an amount of about 9% (w/v).

In embodiments, the methods further comprise drying the article. In embodiments, the article is dried at room temperature, freeze-dried, or dried in a heated oven.

Further aspects of the disclosure may become apparent to those skilled in the art from a review of the following detailed description, taken in conjunction with the examples and appended claims. While the invention is susceptible to embodiments in various forms, described herein are specific embodiments of the invention with the understanding that the disclosure is illustrative, and is not intended to limit the invention to specific embodiments described herein.

### BRIEF DESCRIPTION OF THE DRAWINGS

FIG. 1A is an image of an agarose hydrogel inoculated with 0.1 mL of 10⁶ CFU/mL and exposed to an article of the disclosure having a coating prepared from a mixture including 12% (w/v) urea hydrogen peroxide (UHP) and 1% (w/v) polycaprolactone (PCL).

FIG. 1B is an image of an agarose hydrogel inoculated with 0.1 mL of 10⁶ CFU/mL and exposed to an article of the disclosure having a coating prepared from a mixture including 6% (w/v) UHP and 1% (w/v) PCL.

FIG. 1C is an image of an agarose hydrogel inoculated with 0.1 mL of 10⁶ CFU/mL and exposed to an article of the disclosure having a coating prepared from a mixture including 1% (w/v) UHP and 1% (w/v) PCL.

FIG. 1D is an image of an agarose hydrogel inoculated with 0.1 mL of 10⁶ CFU/mL and exposed to an article having a coating prepared from a mixture including 1% (w/v) PCL.

FIG. 1E is an image an agarose hydrogel inoculated with 0.1 mL of 10⁶ CFU/mL.

FIG. 2A is a graph of the inhibition zone of S. Aureus over 16 days of exposure to an article of the disclosure having a coating prepared from a mixture of PCL and UHP.

FIG. 2B is a graph of the inhibition zone of S. Aureus over 16 days of exposure to an article of the disclosure having a coating prepared from a mixture of PCL and calcium peroxide (CPO).

FIG. 3A is an image of a T-junction microfluidic chip used to prepare the polymer matrices of the disclosure as particles.

FIG. 3B is an image of the polymer matrices of the disclosure as CPO/poly (lactic-co-glycolic) acid (PLGA) particles, prepared using ImageJ software.

FIG. 3C is a graph of the size distribution of polymer matrices of the disclosure prepared as CPO/PLGA particles.

FIG. 3D is an optical image of PLGA particles without CPO (upper) and CPO/PLGA particles (lower).

FIG. 3E is a scanning electron microscopy (SEM) image of a PLGA particle without CPO (scale=10 μm).

FIG. 3F is an SEM image of a PLGA particle without CPO (scale=100 μm).

FIG. 3G is an SEM image of a CPO/PLGA particle where CPO loading was approximately 15 wt % (scale=10 μm).

FIG. 3H is an SEM image of a CPO/PLGA particle where CPO loading was approximately 15 wt % (scale=100 μm).

FIG. 4A is a graph of the change in peroxide concentration over 14 days with CPO/PLGA particles provided in concentrations of 10, 30, and 50 mg/mL.

FIG. 4B is a graph of the change in peroxide concentration over 14 days with CPO/PLGA particles having diameters of 30, 50, and 70 μm.

FIG. 4C is a graph of the change in peroxide concentration over 14 days with CPO/PLGA particles having a CPO loading of 10, 15, and 20 wt %.

FIG. 4D is a graph of the change in peroxide concentration over 14 days of with CPO/PLGA particles with free CPO in solution in concentrations of 0, 0.25, 0.5, and 1 mg/mL.

FIG. 4E is a graph of the change in peroxide concentration over 14 days with CPO/PLGA particles in a concentration of 30 mg/mL with and without 0.5 mg/ml of free CPO.

### DETAILED DESCRIPTION

Hydrogen peroxide is a potent antimicrobial (e.g., antibacterial, antifungal, antiviral, etc.) compound. Advantageously, the articles of the disclosure are capable of effectively inactivating and disinfecting microbes (e.g., viruses, bacteria, fungi, etc.) for an extended period of time by generating small amounts of hydrogen peroxide (i.e., an oxidant) in situ. The articles, via the polymer matrix including a hydrogen peroxide releasing compound can slowly produce and diffuse hydrogen peroxide from the article and into the surrounding environment when exposed to moisture or humidity. The polymer matrices of the disclosure, which can be provided, for example, as coatings and/or particles, can be useful in preparing articles such as filters, bags or pouches, lining materials (e.g., interior linings of bags or containers), medical devices, and bedding materials.

### Articles of the Disclosure

Provided herein are articles having antimicrobial properties. The articles include a substrate and a polymer matrix in contact therewith. The polymer matrix includes a hydrogen peroxide releasing compound. The hydrogen peroxide releasing compound is dispersed within the polymer matrix.

### Substrates

The articles of the disclosure include a substrate. The substrate can be a porous substrate or nonporous substrate. In general, the substrate can include any material that does not undergo significant mechanical friction, as this can cause the polymer matrix to wear over time, and is in need of antimicrobial properties.

In embodiments, the substrate is a porous substrate, such as a woven material or a nonwoven material. The term “porous” refers to the permeability of the substrate material and means that the substrate is susceptible to the passage of liquids, gases, and/or microbes. Substrates of various porosity can be used, and as would be understood by those of skill in the art, as porosity of the substrate increases, the amount of and/or size of gaseous, liquid, or microbial particles that can pass through increases. As used herein, a “woven material” refers to a material that is prepared by interlacing two or more materials (e.g., fibers, threads, fabrics, etc.) at right angles to one another. As would be appreciated by those of skill in the art, woven materials are often made using a loom and are made of many individual fibers or threads on a warp and a weft. An example of a woven material is a textile.

As used herein, a “nonwoven material,” refers to a material in which materials (e.g., fibers, threads, fabrics, etc.) are arranged (e.g., by a carding process) and bonded to each other. Methods of preparing nonwoven materials from fibers are well known in the art, for example, as described in Nonwoven Fabrics Handbook, prepared by lan Butler, edited by Subhash Batra et al., Printing by Design, 1999, herein incorporated by reference in its entirety.

Examples of materials that can be used to form a woven material or a nonwoven material include, but are not limited to, polypropylene fiber, polyacrylic acid fiber, polyurethane fiber, polyester fiber, polyethylene fiber (e.g., high density polyethylene and low density polyethylene), fiberglass fiber, cellulose (e.g., cotton), wood pulp, fluff pulp, abaca, hemp, jute, flax, ramie, sisal, bagasse, banana fiber, lacebark, silk, sinew, catgut, wool, sea silk, mohair, angora, cashmere, collagen, actin, nylon, rayon, bamboo fiber, modal, diacetate fiber, triacetate fiber, copolyester fiber, viscose, polylactide, polyethylene terephthalate (e.g., Dacron), and combinations thereof. As used herein, the term “fiber” encompasses nanofibers and microfibers. In embodiments, the substrate includes a nonwoven material including one or more of cellulose (e.g., cotton), polypropylene fiber, and polyacrylic acid fiber. In embodiments, the polyacrylic acid fiber includes a polyacrylic acid-silver nanofiber. In embodiments, the substrate includes a woven material including one or more of cellulose (e.g., cotton), polypropylene fiber, and polyacrylic acid fiber. In embodiments, the woven material includes a polyacrylic acid-silver nanofiber.

In embodiments, the substrate is a nonporous substrate. The term “nonporous” relates to the permeability of the substrate, and means that there is no appreciable passage of liquids, gases, and/or microbes through the substrate. Nonporous substrates can include, but are not limited to, materials such as glass, plastic, ceramic, stone, or metal. In embodiments, the nonporous substrate includes a polymeric material, a metal alloy, or both. Suitable polymeric materials for use in nonporous substrates include, for example, polyethylene resins, such as high-density polyethylene, low-density polyethylene, and polyethylene terephthalate. Suitable metal alloys for use in nonporous substrates include, for example, steel, stainless steel, brass, bronze, and cast iron.

### Polymer Matrix

The articles of the disclosure include a polymer matrix in contact with the substrate. The polymer matrices include a biodegradable and/or a non-biodegradable polymer. The polymer matrices further include a hydrogen peroxide releasing compound dispersed within (e.g., throughout) the polymer matrix such that they are physically trapped (or entrapped) therein. In general, because the exposure of hydrogen peroxide releasing compounds to water can prompt the dissociation and release of the hydrogen peroxide, the hydrogen peroxide compound of the disclosure is advantageously incorporated into the polymer matrix to delay its dissociation and release and prolong its lifespan. Without intending to be bound by theory, there are no particular electrostatic, covalent, ionic, etc. interactions expected between the polymers of the matrix and the hydrogen peroxide releasing compounds. Rather, particles of the hydrogen peroxide releasing compounds can be prepared and randomly distributed in polymer matrix through mixing. In embodiments, the polymer matrix includes a homogeneous distribution of the hydrogen peroxide releasing compounds. As used herein, the term “homogeneous” or any variety thereof means that the hydrogen peroxide releasing compound (or whichever component with which the descriptor is used) is uniformly distributed such that the distances between each of the hydrogen peroxide releasing compounds vary by no more than about 10%, for example about 10, 9, 8, 7, 6, 5, 4, 3, 2, or 1%. For example, the polymer matrix can include a homogeneous distribution of the hydrogen peroxide releasing compounds such that the distances between each of the hydrogen peroxide releasing compounds in the polymer matrix vary by no more than about 10%. The particle size of the hydrogen peroxide releasing compounds can depend on the particular application in which the articles of the disclosure are used.

The polymer matrices can be provided as a coating or as a particle (e.g., powders, beads, etc.). In embodiments, the polymer matrix is provided as a coating, and the coating is in contact with the substrate, as described herein. In embodiments, the polymer matrix is provided as a particle, and the particle is in contact with the substrate. As used herein, the term “in contact with” means that the polymer matrix can be coated on, weaved in, embedded in, entangled in (e.g., mechanically entangled), encapsulated by, or otherwise associated with the substrate. For example, in embodiments, the polymer matrix is provided as a coating for the substrate (e.g., as a coating for the substrate as a whole, or as a coating for the individual fibers of the substrate where the substrate is a porous substrate). In embodiments, the polymer matrix is provided as a particle and is woven, entangled, embedded, or otherwise associated with the substrate. For example, the particle can be distributed homogenously throughout a porous substrate via the weaving or carding process for woven and nonwoven substrates, respectively (e.g., such that the distances between each of the particles in the porous substrate vary no more than 10%). In embodiments, the polymer matrix is provided as a particle and is encapsulated or contained within a container made using the substrate (e.g., a pouch or sachet). For example, a sealed porous container (prepared from the substrate material) can encapsulate or contain the polymer matrices (prepared as particles) and be placed within a larger container such that the polymer matrices can release hydrogen peroxide gas through the porous substrate material and into the larger container to disinfect items (e.g., masks, etc.) placed within the larger container.

### Polymer

As described herein, the polymer matrix can include a biodegradable polymer and/or a nonbiodegradable polymer. In embodiments, the polymer matrix includes a biodegradable polymer. In embodiments, the polymer matrix includes a nonbiodegradable polymer. In embodiments, the polymer matrix includes a biodegradable polymer and a nonbiodegradable polymer. In general, the polymer matrix can include a polymer that can break down over time to allow the dissociation and release of the hydrogen peroxide compound. Without intending to be bound by theory, it is believed that the break down of a biodegradable polymer is facilitated by environmental factors, such as, water/humidity, pH, light, and temperature. Without intending to be bound by theory, it is believed that the break down of nonbiodegradable polymers can be facilitated by the hydrogen peroxide source that is dispersed throughout the polymer matrix. It is believed that the hydrogen peroxide source can degrade the polymer to provide a pore or channel until a path to the surface of the polymer matrix is formed and the hydrogen peroxide sources is exposed to the environment, allowing the dissociation and release of the hydrogen peroxide compound.

In embodiments, the polymer matrices of the disclosure include a biodegradable polymer. The biodegradable polymer can include, for example, polyglycolic acid (PGA), polycaprolactone (PCL), polylactic acid (PLA), polyurethane (PU), a polyester, poly (lactic-co-glycolic acid) (PLGA), or combinations thereof. In embodiments, the biodegradable polymer includes polyglycolic acid (PGA). In embodiments, the biodegradable polymer include polycaprolactone (PCL). In embodiments, the biodegradable polymer includes polylactic acid (PLA). In embodiments, the biodegradable polymer includes polyurethane (PU). In embodiments, the biodegradable polymer includes a polyester. In embodiments, the biodegradable polymer include poly (lactic-co-glycolic acid) (PLGA).

In embodiments, the polymer matrices of the disclosure include a nonbiodegradable polymer.

The polymer can be present in an amount of about 5 wt % to about 95 wt %, for example at least about 5, 6, 7, 8, 9, 10, 15, 20, 25, 30, 35, 40, 45, 50, 55, or 60 wt % and/or up to about 20, 30, 40, 45, 50, 55, 60, 65, 70, 75, 80, 85, 90, 91, 92, 93, 94, or 95 wt %, based on the total weight of the polymer matrix. For example, the total amount of any biodegradable polymer and/or nonbiodegradable polymer can be in a range of about 5 wt % to about 90 wt %, about 10 wt % to about 90 wt %, about 10 wt % to about 80 wt %, about 15 wt % to about 80 wt %, about 15 wt % to about 75 wt %, about 20 wt % to about 75 wt %, about 20 wt % to about 70 wt %, about 25 wt % to about 70 wt %, about 25 wt % to about 65 wt %, about 30 wt % to about 60 wt %, about 35 wt % to about 60 wt %, about 35 wt % to about 55 wt %, about 40 wt % to about 55 wt %, or about 40 wt % to about 50 wt %, based on the total weight of the polymer matrix. In embodiments, the polymer is present in an amount of about 90 wt %, based on the total weight of the polymer matrix. In embodiments, the polymer is present in an amount of about 80 wt %, based on the total weight of the polymer matrix. In embodiments, the polymer is present in an amount of about 60 wt %, based on the total weight of the polymer matrix. In embodiments, the polymer is present in an amount of about 50 wt %, based on the total weight of the polymer matrix. In embodiments, the polymer is present in an amount of about 40 wt %, based on the total weight of the polymer matrix. In embodiments, the polymer is present in an amount of about 14 wt %, based on the total weight of the polymer matrix. In embodiments, the polymer is present in an amount of about 8 wt %, based on the total weight of the polymer matrix.

Without intending to be bound by theory, it is believed that as the amount of polymer within the polymer matrix decreases, the hydrogen peroxide releasing compound can dissociate more readily due to increased exposure to the environment. As a result, it is expected that the lifespan of the hydrogen peroxide releasing compound will decrease as the amount of polymer in the polymer matrix decreases. This may be advantageous in embodiments where long-term activity is not necessary (e.g., in single-or limited-use face masks, or linings of or containers within packages containing single-or limited-use face masks). In contrast, it is expected that as the amount of polymer within the polymer matrix increases, the hydrogen peroxide releasing compound is activated more slowly due to decreased exposure to the environment. For example, increased amounts of polymer can decrease the interconnected porosity of the polymer matrix, thereby delaying the exposure of the hydrogen peroxide releasing compound to environmental humidity and delaying the release of hydrogen peroxide therefrom. As a result, it is expected that the lifespan of the hydrogen peroxide releasing compound will increase as the amount of polymer in the polymer matrix increases. This may be advantageous in embodiments where long-term activity is desired (e.g., in HEPA filters, HVAC filters, A/C filters, biohazard waste containers, etc.).

### Hydrogen Peroxide Releasing Compound

The polymer matrices of the disclosure include a hydrogen peroxide releasing compound. The hydrogen peroxide releasing compounds of the disclosure can release at least about 100 ppm hydrogen peroxide into the surrounding environment, which is an amount that has been shown to kill microbes (e.g., bacteria) effectively.

The hydrogen peroxide releasing compound can include hydrogen peroxide, itself, as well as other suitable hydrogen peroxide releasing compounds. In embodiments, the hydrogen peroxide releasing compound is hydrogen peroxide. In embodiments, the hydrogen peroxide releasing compound includes a metal peroxide. Suitable examples of metal peroxides include, but are not limited to, calcium peroxide, magnesium peroxide, silver peroxide, copper peroxide, and zinc peroxide. A single metal peroxide or a mixture of two or more different metal peroxides can be used. Some metal peroxides, e.g., zinc peroxide, silver peroxide, etc., can exhibit dual action in which both the metal and the hydrogen peroxide released therefrom have antimicrobial properties.

In embodiments, the hydrogen peroxide releasing compound includes urea-hydrogen peroxide (UHP, i.e., carbamide peroxide). Urea-hydrogen peroxide is commonly used for disinfection and bleaching proposes in the cosmetic and pharmaceutical industries and also for dental care and mouth hygiene. UHP can be provided as a crystalline powder that can dissolve in water and produces urea and hydrogen peroxide according to Formula (1):

CH₆N₂O₃→CH₄N₂O +H₂O₂ (1)

Urea is a colorless, odorless solid, highly soluble in water, and substantially non-toxic (LD₅₀ is about 15 g/kg in rats). Urea is widely used as a skin medication and applied to the skin to treat dryness and itching. The hydrogen peroxide release product is the source of hydroxyl radicals (HO·) that impart the antimicrobial properties of the articles of the disclosure. Hydrogen peroxide has low stability and breaks down to water and oxygen according to Formula 2:

2H₂O₂→2H₂O+O₂ (2)

In addition to activation via dissociation and release, UHP can additionally be activated due to its high water solubility. If the concentration of a water-soluble hydrogen peroxide releasing compound, such as UHP, is high enough (e.g., at least 5 wt % based on total weight of the UHP and the polymer) and at a sufficient humidity, then the compound can be activated by dissolving (e.g., via humidity in the environment) and releasing hydrogen peroxide to an exposed surface of the polymer matrix. This phenomenon, in which the hydrogen peroxide releasing compound acts as a porogen, can create access to the hydrogen peroxide releasing compounds present deeper within the polymer matrix and/or the substrate. In embodiments, the hydrogen peroxide releasing compound includes UHP and a metal peroxide.

The hydrogen peroxide releasing compound can be present in the polymer matrix in an amount of about 0.1 wt % to about 99.9 wt %, for example at least about 0.1, 0.2, 0.5, 1, 2, 3, 4, 5, 6, 7, 8, 9, 10, 15, 20, 25, 30, 35, 40, 45, 50, 55, or 60 wt % and/or up to about 20, 30, 40, 45, 50, 55, 60, 65, 70, 75, 80, 85, 90, 91, 92, 93, 94, 95, 96, 97, 98, 99, 99.5, or 99.9 wt %, based on the total weight of the polymer matrix. For example, the hydrogen peroxide releasing compound can be present in an amount of about 0.1 wt % to about 99.9 wt. %, 5 wt % to about 90 wt %, about 10 wt % to about 90 wt %, about 10 wt % to about 80 wt %, about 15 wt % to about 80 wt %, about 15 wt % to about 75 wt %, about 20 wt % to about 75 wt %, about 20 wt % to about 70 wt %, about 25 wt % to about 70 wt %, about 25 wt % to about 65 wt %, about 30 wt % to about 60 wt %, about 35 wt % to about 60 wt %, about 35 wt % to about 55 wt %, about 40 wt % to about 55 wt %, or about 40 wt % to about 50 wt %, based on the total weight of the polymer matrix. In embodiments, the hydrogen peroxide releasing compound is present in an amount of about 5 wt % to about 95 wt %. In embodiments, the hydrogen peroxide releasing compound is present in an amount of about 92 wt %, based on the total weight of the polymer matrix. In embodiments, the hydrogen peroxide releasing compound is present in an amount of about 86 wt %, based on the total weight of the polymer matrix. In embodiments, the hydrogen peroxide releasing compound is present in an amount of about 60 wt %, based on the total weight of the polymer matrix. In embodiments, the hydrogen peroxide releasing compound is present in an amount of about 50 wt %, based on the total weight of the polymer matrix. In embodiments, the hydrogen peroxide releasing compound is present in an amount of about 40 wt %, based on the total weight of the polymer matrix. In embodiments, the hydrogen peroxide releasing compound is present in an amount of about 20 wt %, based on the total weight of the polymer matrix. In embodiments, the hydrogen peroxide releasing compound is present in an amount of about 10 wt %, based on the total weight of the polymer matrix.

Without intending to be bound by theory, it is believed that as the amount of hydrogen peroxide releasing compound within the polymer matrix increases, the antimicrobial lifespan of the article can be increased. For example, in embodiments wherein the polymer matrix includes biodegradable polymers that quickly degrade, such as PLGA, in an amount of up to about 50 wt %, based on the total weight of the polymer matrix, the articles can have a sustained release of hydrogen peroxide for at least about three weeks. In embodiments wherein the polymer matrix includes biodegradable polymers that degrade more slowly (e.g., over several days or weeks), such as PCL, in an amount of about 50 wt %, based on the total weight of the polymer matrix, the articles may require increased amounts of hydrogen peroxide releasing compound to generate sufficient interconnected porosity within the polymer matrix to allow for effective release of the hydrogen peroxide upon dissociation. Where slower degradable polymers and/or nonbiodegradable polymers are used, it can be beneficial to include hydrogen peroxide releasing compounds having increased particle sizes (e.g., at least about 0.3 μm) such that presence of the hydrogen peroxide releasing compound can create porosity within the polymer matrix and article to allow for release of the compound. Where the polymer is a biodegradable polymer that degrades more quickly and/or where less polymer is present within the matrix, the particle size of the hydrogen peroxide releasing compound is less restricted, as the matrix has sufficient porosity to result in effective release of hydrogen peroxide.

The hydrogen peroxide releasing compound and the polymer can be present in the polymer matrix in a weight ratio of about 1:19 to about 19:1, for example at least about 1:19, 1:18, 1:15, 1:11, 1:10, 1:9, 1:7, 1:5, 1:4, 1:2, 2:3, or 1:1, and/or up to about 1:5, 1:2, 1:1, 2:1, 3:2, 5:1, 6:1, 8:1, 10:1, 11:1, 15:1, 17:1, or 19:1. For example, the hydrogen peroxide releasing compound and the polymer can be present in the polymer matrix in a weight ratio of about 1:10 to about 15:1, about 1:9 to about 11:1, about 1:4 to about 6:1, about 2:3 to about 3:2, or about 1:1. In embodiments, hydrogen peroxide releasing compound and the polymer can be present in a weight ratio of about 1:9. In embodiments, hydrogen peroxide releasing compound and the polymer can be present in a weight ratio of about 1:4. In embodiments, hydrogen peroxide releasing compound and the polymer can be present in a weight ratio of about 2:3. In embodiments, hydrogen peroxide releasing compound and the polymer can be present in a weight ratio of about 1:1. In embodiments, hydrogen peroxide releasing compound and the polymer can be present in a weight ratio of about 3:2. In embodiments, hydrogen peroxide releasing compound and the polymer can be present in a weight ratio of about 6:1. In embodiments, hydrogen peroxide releasing compound and the polymer can be present in a weight ratio of about 11:1.

In embodiments, the polymer matrix is free of any components other than the polymer and the hydrogen peroxide releasing compound. As used herein, “free of any components other than the polymer and the hydrogen peroxide releasing compound” means that the polymer matrix does not include any non-polymer and non-hydrogen peroxide releasing components (e.g., additives, catalysts, dispersants, etc.) such that at least 90% by weight of the polymer matrix is attributable to the polymer and the hydrogen peroxide releasing compounds. In embodiments, at least 90, 95, 97, 98, 99, 99.5, 99.9, or 100 wt % of the polymer matrix is attributable to the polymer and the hydrogen peroxide releasing compound, alone. As would be appreciated by those skilled in the art, the polymer matrix may include residual solvent leftover from the preparation of the polymer matrix, as described herein, where the solvent may be trapped in the matrix and unable to readily evaporate.

In embodiments, the hydrogen peroxide releasing compound includes UHP and the polymer includes a biodegradable polymer. In embodiments, the hydrogen peroxide releasing compound includes UHP and the polymer includes a biodegradable polymer including PCL, PLGA, or both.

In embodiments, the hydrogen peroxide releasing compound includes UHP and the polymer includes PCL. In embodiments, the UHP is present in an amount of about 92 wt %, based on the total weight of the polymer matrix, and the PCL is present in an amount of about 8 wt %, based on the total weight of the polymer matrix. In embodiments, the UHP is present in an amount of about 86 wt %, based on the total weight of the polymer matrix, and the PCL is present in an amount of about 14 wt %, based on the total weight of the polymer matrix. In embodiments, the UHP is present in an amount of about 60 wt %, based on the total weight of the polymer matrix, and the PCL is present in an amount of about 40 wt %, based on the total weight of the polymer matrix. In embodiments, the UHP is present in an amount of about 50 wt %, based on the total weight of the polymer matrix, and the PCL is present in an amount of about 50 wt %, based on the total weight of the polymer matrix. In embodiments, the UHP is present in an amount of about 40 wt %, based on the total weight of the polymer matrix, and the PCL is present in an amount of about 60 wt %, based on the total weight of the polymer matrix. In embodiments, the UHP is present in an amount of about 20 wt %, based on the total weight of the polymer matrix, and the PCL is present in an amount of about 80 wt %, based on the total weight of the polymer matrix. In embodiments, the UHP is present in an amount of about 10 wt %, based on the total weight of the polymer matrix, and the PCL is present in an amount of about 90 wt %, based on the total weight of the polymer matrix.

In embodiments, the hydrogen peroxide releasing compound includes UHP and the polymer includes PLGA. In embodiments, the UHP is present in an amount of about 92 wt %, based on the total weight of the polymer matrix, and the PLGA is present in an amount of about 8 wt %, based on the total weight of the polymer matrix. In embodiments, the UHP is present in an amount of about 86 wt %, based on the total weight of the polymer matrix, and the PLGA is present in an amount of about 14 wt %, based on the total weight of the polymer matrix. In embodiments, the UHP is present in an amount of about 60 wt %, based on the total weight of the polymer matrix, and the PLGA is present in an amount of about 40 wt %, based on the total weight of the polymer matrix. In embodiments, the UHP is present in an amount of about 50 wt %, based on the total weight of the polymer matrix, and the PLGA is present in an amount of about 50 wt %, based on the total weight of the polymer matrix. In embodiments, the UHP is present in an amount of about 40 wt %, based on the total weight of the polymer matrix, and the PLGA is present in an amount of about 60 wt %, based on the total weight of the polymer matrix. In embodiments, the UHP is present in an amount of about 20 wt %, based on the total weight of the polymer matrix, and the PLGA is present in an amount of about 80 wt %, based on the total weight of the polymer matrix. In embodiments, the UHP is present in an amount of about 10 wt %, based on the total weight of the polymer matrix, and the PLGA is present in an amount of about 90 wt %, based on the total weight of the polymer matrix.

When the article includes a porous substrate, the polymer matrix can be applied in such an amount that the pores of the substrate are not substantially filled, blocked, or clogged. That is, the polymer matrix can be applied such that liquids, gases, and/or microbes are able to permeate the polymer matrix-containing substrate. In some embodiments, when the article includes a porous substrate, the polymer matrix can be applied in such an amount that the pores of the substrate are no longer permeable to one or more of liquids, gases, and/or microbes, and remain permeable to one or more of liquids, gases, and/or microbes. In embodiments, when the article includes a porous substrate, the polymer matrix can be applied in such an amount that the pores of the substrate are impermeable to liquids, gases, and microbes. For example, the polymer matrix can be applied to a porous substrate in an amount such that the weight of the final article increases by at least about 4, 5, 6, 7, 8, 9, or 10 times, as compared to the initial weight of the substrate. Such an increase in the weight of the article, as compared to the initial weight of the substrate, can be attributed to the high surface area of the porous substrate in combination with the density of the polymer matrix or the viscosity of the solution used to prepare the polymer matrix (e.g., where the polymer matrix is applied as a coating). For example, the high surface area can provide for increased retention of a viscous coating solution within the pores of the porous substrate.

### Article Properties

As described herein, the articles of the disclosure exhibit antimicrobial properties. As used herein, the term “antimicrobial properties,” encompasses antibacterial, antiviral, and antifungal properties. In embodiments, the article is an antimicrobial article. In embodiments, the antimicrobial article is effective against one or more of a fungus, gram-positive bacteria, gram-negative bacteria, spore of bacteria, and a virus. As used herein, the term “effective against” means that the article can kill or deactivate one or more of a fungus, a gram-negative bacteria, a gram-positive bacteria, a spore of bacteria, and a virus. An article can be effective against one or more of a fungus, a gram-negative bacteria, a gram-positive bacteria, a spore of bacteria, and a virus when the article kills or deactivates at least about 90% of the fungus, gram-positive bacteria, gram-negative bacteria, spore of bacteria, and/or virus. For example, the article can be effective when the article kills or deactivates at least about 90, 95, 97, 99, 99.5, 99.9, or 100% of the fungus, gram-positive bacteria, gram-negative bacteria, spore of bacteria, and/or virus. The efficiency of the article against these microbes can be measured by counting the number of microbes after exposure to the article in accordance with methods known in the art and available from the U.S. Environmental Protection Agency or the U.S. Federal Drug Administration, such as EPA MLB SOP MB-37-00.

In embodiments, the article is an antimicrobial article that is effective against a fungus. Examples of fungi that may be susceptible to the articles of the disclosure include, but are not limited to, those of the Candida, Cryptococcus, Blastomyces, Paracoccidioides, Coccidioides, Histoplasma, Fusarium, Aspergillus, and Pneumocystis genera. In embodiments, the fungs includes one or more of Candida albicans, Candida auris, Cryptococcus gattii, Cryptococcus neoformans, Blastomyces dermatitidis, Paracoccidioides brasiliensis, Paracoccidioides lutzii, Coccidioides immitis, Coccidioides posadasii, Histoplasma capsulatum, Fusarium solani, Fusarium oxysporum, Fusarium verticillidioidis, Fusarium monoliforme, Aspergillus niger, Aspergillus flava, Aspergillus fumigatus, Aspergillis parasiticus, and Pneumocystis jirovecii. In embodiments, the articles of the disclosure are effective against Aspergillus flava, Aspergillis parasiticus, or both. Each of A. flavus and A. parasiticus are the primary producers of aflatoxins, one of the leading causes of liver cancer in the world. Aflatoxins are a family of toxins produced by certain fungi that are found on agricultural crops such as maize (corn), peanuts, cottonseed, and tree nuts. They also can contaminate and grow on air filters (e.g., air conditioner (AC) filters), thus producing and releasing aflatoxins into the air.

In embodiments, the article is an antimicrobial article that is effective against a gram-negative bacteria. Examples of gram-negative bacteria that may be susceptible to the articles of the disclosure include, but are not limited to, Escherichia coli, Pseudomonas aeruginosa, Klebsiella pneumoniae, Klebsiella oxytoca, Klebsiella terrigena, Klebsiella planticola, Acinetobacter baumannii, Acinetobacter calcoaceticus, and Mycobacterium tuberculosis. In embodiments, the articles of the disclosure are effective against E. coli, P. aeruginosa, or both. In embodiments, the article is an antimicrobial article that is effective against a gram-positive bacteria. Examples of gram-positive bacteria that may be susceptible to the articles of the disclosure include, but are not limited to, Staphylococcus aureus, methicillin-resistant Staphylococcus aureus (MRSA), Clostridium difficile, and vancomycin-resistant Enterococcus (VRE). In embodiments, the articles of the disclosure are effective against MRSA, VRE, or both. The articles of the disclosure can be bacteriostatic. That is, the articles of the disclosure can inhibit growth of gram-negative and/or gram-positive bacteria. The articles of the disclosure can be bactericidal. That is, the articles of the disclosure can kill gram-negative and/or gram-positive bacteria.

In embodiments, the article is an antimicrobial article that is effective against a virus. The article can be effective against enveloped viruses, non-enveloped viruses, or both. Examples of viruses that may be susceptible to the articles of the disclosure include, but are not limited to those that cause viral diseases such as COVID-19 (e.g., SARS-COV-2 and variants), chickenpox (e.g., VZV or HHV-3), influenza (e.g., H1N1, H3N2, etc.), herpes (e.g., HSV-1, HSV-2, etc.), human immunodeficiency virus (HIV/AIDS) (e.g., HIV-1, HIV-2, etc.), human papillomavirus (HPV) (e.g., HPV-16, HPV-18, HPV-6, HPV-11, etc.), infectious mononucleosis (e.g., EBV or HHV-4), mumps, measles, rubella, shingles (e.g., VZV or HHV-3), viral gastroenteritis, viral hepatitis, viral meningitis, and viral pneumonia. In embodiments, the articles are effective against viruses that cause viral diseases such as COVID-19, influenza, or both.

As described herein, the articles of the disclosure include dispersed or entrapped hydrogen peroxide releasing compounds within a polymer matrix. Therefore, and advantageously, the articles of the disclosure can delay the onset of the dissociation and release of hydrogen peroxide and water from the hydrogen peroxide releasing compound. In embodiments, the hydrogen peroxide releasing compounds can be stable until they are exposed to a threshold level of moisture or water, such as a relative humidity (RH) of at least about 5, 6, 7, 8, 9, 10, 15, 20, 25, 30, 35, 40, 45, 50, 55 or 60% and/or up to about 25, 30, 35, 40, 45, 50, 55, 60, 65, 70, 75, 80, 85, 90, 95, 96, 97, 98, 99, or 100%. For example, hydrogen peroxide can be released from the article (e.g., upon dissociation and release of hydrogen peroxide and water from the hydrogen peroxide releasing compound) when the article is exposed to a relative humidity of about 5% to about 100%, about 5% to about 95%, about 5% to about 90%, about 10% to about 90%, about 25% to about 75%, about 30% to about 100%, about 5% to about 25%, about 50% to about 80%, or about 90% to about 100%. In embodiments, hydrogen peroxide is released from the hydrogen peroxide releasing compound when the article is exposed to a relative humidity of at least about 5%. In embodiments, hydrogen peroxide is released from the hydrogen peroxide releasing compound when the article is exposed to a relative humidity of about 5% to about 25%. In embodiments, hydrogen peroxide is released from the hydrogen peroxide releasing compound when the article is exposed to a relative humidity of at least about 30%. In embodiments, hydrogen peroxide is released from the hydrogen peroxide releasing compound when the article is exposed to a relative humidity of about 90% to about 100%. The relative humidity can be measured in accordance with methods known in the art.

The hydrogen peroxide releasing compound, the polymer, and the relative amounts thereof can be particularly selected depending on the end-use and application of the article. For example, for articles that will be used in high humidity environments, the hydrogen peroxide releasing compound, the polymer, and the relative amounts thereof can be tailored to maximize the lifespan and sustained onset of activation. Similarly, for articles that will be used in low humidity environments, the hydrogen peroxide releasing compound, the polymer, and the relative amounts thereof can be tailored to maximize the lifespan and the delay of onset of activation. Without intending to be bound by theory, where the environment is a high humidity environment (e.g., at least about 50% RH), then more polymer can be incorporated into the polymer matrix to reduce or delay the dissociation and release of hydrogen peroxide from the article. Similarly, where the environment is a low-humidity environment (e.g., less than about 50% RH), less polymer and more hydrogen peroxide releasing compound can be incorporated to prolong the dissociation and release of hydrogen peroxide.

The articles of the disclosure can be, or can be incorporated into, a filter, a container material, a lining material, a medical device, or a bedding material. For example, the article can be a filter, a container (e.g., a bag, a sachet, a pouch, etc.), a lining material, a medical device, or a bedding material, where the article, as is, can provide the desired properties. Alternatively, the article can be incorporated into a filter, a container (e.g., a bag, a sachet, a pouch, etc.), a lining material, a medical device, or a bedding material, where the article, as is, can be provided as at least one layer of the final product (e.g., a layer within a HEPA filter, where the other layers of the HEPA filter do not include the article or polymer matrix of the disclosure, or a layer within a container material, where the other layers of the container material do not include the article or polymer matrix of the disclosure).

In embodiments, the article is or is incorporated into a filter. Examples of suitable filters include, but are not limited to, face masks, respirators, HVAC filters, vacuum filters, and HEPA filters. In embodiments, the filter is a face mask, such as an N95 face mask, or a woven, cloth face mask. In embodiments, the filter is a respirator. In embodiments, the filter is an HVAC filter. In embodiments, the filter is a vacuum filter. In embodiments, the filter is a HEPA filter. Advantageously, because UHP is relatively nontoxic, the articles of the disclosure (e.g., at least when the hydrogen peroxide releasing compound is or includes UHP) can be used in face masks without exposing the consumer to harmful or toxic materials when tailored to include a suitable ratio of hydrogen peroxide releasing compound and polymer. Additionally, or alternatively, to further limit toxicity concerns (e.g., to comply with OSHA's standard that hydrogen peroxide be less than 1 ppm in breathing air), the hydrogen peroxide releasing compound can include zinc peroxide and/or calcium peroxide which release lower concentrations of hydrogen peroxide and have dual-antimicrobial functionality. The hydrogen peroxide releasing compound in such cases can be activated by the humidity present in the breath of the consumer.

In embodiments, the article is or is incorporated into a bedding material. Examples of suitable bedding material includes a mattress, a mattress pad, a sheet, a pillow, a pillow case, a comforter, or a duvet cover. Notably, Aspergillus fumigatus is a fungal species most commonly found in pillows, which can lead to the onset of Aspergillosis, the leading cause of death in leukemia and bone marrow transplants. Therefore, use of the articles of the disclosure in bedding materials, such as pillows and pillow cases can advantageously protect hospital residents, without exposing the residents to additional toxins or harmful materials. In embodiments, the bedding material is a mattress. In embodiments, the bedding material is a mattress pad. In embodiments, the bedding material is a sheet. In embodiments, the bedding material is a pillow. In embodiments, the bedding material is a pillow case. In embodiments, the bedding material is a comforter. In embodiments, the bedding material is a duvet cover.

In embodiments, the article is or is incorporated into a medical device. Examples of suitable medical devices include, but are not limited to, disinfectant wipes, wound dressings, or biohazard reservoirs. Examples of biohazard reservoirs include, for example, waste bins, waste basins, sharps containers, waste bottles, trash bags, and the like. The articles can be incorporated into, for example, single-use waste bins, waste bags, or waste bottles which can allow for the near instantaneous neutralization of the contaminated materials placed therein. Advantageously, such an article can protect patients and personnel from possible infection by accidental spill or release of any contaminated fluids, solids, or aerosols generated from these reservoirs.

In embodiments, the article is or is incorporated into a lining material. For example, the articles of the disclosure can be used or incorporated into a lining material on an interior surface of a bag, package, or container. In such embodiments, the lining can be used to release hydrogen peroxide into the package to preserve and disinfect the contents of the package. For example, it is envisaged that the lining can be used on the interior of a resealable package containing a number face masks (e.g., N95 masks or woven, cloth masks) such that after the contents are initially exposed to the environment (e.g., after the package is opened for the first time and thereafter resealed) the lining material can release hydrogen peroxide into the package to disinfect the remaining contents. As the lining of the package does not have significant contact with the user (e.g., skin, hair, nails, etc.), the amount of hydrogen peroxide in the lining material would not be limited by the safety levels set for human contact, and can still provide antimicrobial properties to the contents of the bag (e.g., face masks).

In embodiments, the article is or is incorporated into a container material. For example, the articles of the disclosure can be used to form sealed containers (e.g., pouches, pods, sachets, bags, etc.) where the substrate (e.g., a porous substrate such as a textile) encapsulates, encloses, or contains the polymer matrices (e.g., provided as particles) therein. The sealed containers can then be placed within a larger container (e.g., a box, bag, etc.) along with other items (e.g., masks, surgical tools, cosmetic tools, makeup, toiletries, etc.) such that the hydrogen peroxide gas, released from the polymer matrix within the container can travel through the substrate material forming the container and into the larger container to disinfect the other items contained therein. In embodiments, the article is or is incorporated into a pouch or a pod that can be punctured or cracked to allow release of hydrogen peroxide gas from the polymer matrices (e.g., provided as particles) provided within the pod. Such an embodiment would allow a consumer to disinfect the contents of a bag (e.g., in which the pod was placed), without spilling the polymer matrix particles into the bag, thereby reducing any skin contact with the particles. In embodiments, the sealed container can be gas permeable and the hydrogen peroxide forming compound can be provided in the sealed container as a loose powder, e.g., wherein the hydrogen peroxide forming compound is provided at greater than 90, 95, 97, 99, 99.5, or 99.9 wt % based on the total weight of the matrix.

In embodiments, the article is or is incorporated into a tool or electronic gadget that has high contact to consumers, such as a mobile phone, a tablet, a computer, cosmetics (e.g., makeup tools, applicators, containers, etc.), utensils (e.g., toothbrushes, spoons, forks, knives, etc.), or minor surgical tools (e.g., tweezers, clippers, speculum, forceps, etc.).

Notably, the articles of the disclosure can release a concentration of hydrogen peroxide into the air that is high enough to neutralize microbes (e.g., bacteria, fungi, viruses, etc.) that are also present in the air. Therefore, and advantageously, the articles of the disclosure may not require that the microbes come into direct contact with the articles of the disclosure in order for the articles to exhibit its antimicrobial properties.

### Methods of Forming Articles

The disclosure further provides methods of forming the articles of the disclosure. The methods include contacting the substrate, as described herein, with a mixture including the hydrogen peroxide releasing compound and the polymer of the disclosure, to provide the article. The methods can further include drying, curing, or otherwise solidifying the mixture on the substrate.

The surfaces of the substrates can be contacted in a number of suitable ways known in the art. For example, where the mixture is provided as a coating (e.g., a liquid mixture or a solution), contacting the surface with the mixture can occur after a discrete substrate has been prepared and is thereafter contacted with the mixture by methods including, but not limited to, dipping the surface in the mixture, brushing the mixture onto the surface, and/or spraying the surface with the mixture. Additionally or in the alternative, where the mixture is provided as a coating (e.g., a liquid mixture, a solution, etc.) or as a particle (e.g., a bead, a powder, etc.), the contacting can occur during the fabrication of the underlying substrate, for example, by methods including but not limited to melt-spinning the substrate material with the mixture, extruding the substrate material with the mixture, weaving the substrate material with the mixture, entangling the substrate material with the mixture, or molding the substrate material with the mixture.

The hydrogen peroxide releasing compound can be present in the mixture in any amount suitable to provide the articles of the disclosure. For example, the hydrogen peroxide releasing compound can be present in the mixture in amount of about 5 wt % to about 95 wt %, for example at least about 5, 6, 7, 8, 9, 10, 15, 20, 25, 30, 35, 40, 45, 50, 55, or 60 wt % and/or up to about 20, 30, 40, 45, 50, 55, 60, 65, 70, 75, 80, 85, 90, 91, 92, 93, 94, or 95 wt %, based on the total combined weight of the hydrogen peroxide releasing compound and the polymer. For example, the hydrogen peroxide releasing compound can be present in an amount of about 5 wt % to about 90 wt %, about 10 wt % to about 90 wt %, about 10 wt % to about 80 wt %, about 15 wt % to about 80 wt %, about 15 wt % to about 75 wt %, about 20 wt % to about 75 wt %, about 20 wt % to about 70 wt %, about 25 wt % to about 70 wt %, about 25 wt % to about 65 wt %, about 30 wt % to about 60 wt %, about 35 wt % to about 60 wt %, about 35 wt % to about 55 wt %, about 40 wt % to about 55 wt %, or about 40 wt % to about 50 wt %, based on the total combined weight of the hydrogen peroxide releasing compound and the polymer. In embodiments, the hydrogen peroxide releasing compound is present in an amount of about 92 wt %, based on the total weight of the hydrogen peroxide releasing compound and the polymer. In embodiments, the hydrogen peroxide releasing compound is present in an amount of about 86 wt %, based on the total weight of the hydrogen peroxide releasing compound and the polymer. In embodiments, the hydrogen peroxide releasing compound is present in an amount of about 60 wt %, based on the total weight of the hydrogen peroxide releasing compound and the polymer. In embodiments, the hydrogen peroxide releasing compound is present in an amount of about 50 wt %, based on the total weight of the hydrogen peroxide releasing compound and the polymer. In embodiments, the hydrogen peroxide releasing compound is present in an amount of about 40 wt %, based on the total weight of the hydrogen peroxide releasing compound and the polymer. In embodiments, the hydrogen peroxide releasing compound is present in an amount of about 20 wt %, based on the total weight of the hydrogen peroxide releasing compound and the polymer. In embodiments, the hydrogen peroxide releasing compound is present in an amount of about 10 wt %, based on the total weight of the hydrogen peroxide releasing compound and the polymer.

The hydrogen peroxide releasing compound can present in the mixture in an amount in a range of about 1% (w/v) to about 20% (w/v), for example at least about 1, 2, 3, 4, 5, 6, 7, 8, 9, 10, 11 or 12% (w/v) and/or up to about 8, 9, 10, 11, 12, 13, 14, 15, 16, 17, 18, 19, or 20% (w/v), such as about 1% (w/v) to about 19% (w/v), about 2% (w/v) to about 18% (w/v) about 3% (w/v) to about 17% (w/v), about 4% (w/v) to about 16% (w/v), about 5% (w/v) to about 15% (w/v), about 6% (w/v) to about 14% (w/v) about 7% (w/v) to about 13% (w/v), about 8% (w/v) to about 12% (w/v), about 10% (w/v) to about 12% (w/v), about 1% (w/v) to about 10% (w/v), about 2% (w/v) to about 8% (w/v), about 4% (w/v) to about 6% (w/v), or about 5% (w/v). In embodiments, the hydrogen peroxide releasing compound is present in the mixture in an amount of about 1% (w/v). In embodiments, the hydrogen peroxide releasing compound is present in the mixture in an amount of about 2% (w/v). In embodiments, the hydrogen peroxide releasing compound is present in the mixture in an amount of about 4% (w/v). In embodiments, the hydrogen peroxide releasing compound is present in the mixture in an amount of about 5% (w/v). In embodiments, the hydrogen peroxide releasing compound is present in the mixture in an amount of about 6% (w/v). In embodiments, the hydrogen peroxide releasing compound is present in the mixture in an amount of about 12% (w/v).

The polymer can be present in the mixture in an amount suitable to provide the

articles of the disclosure. For example, the polymer can be present in the mixture in an amount of about 5 wt % to about 95 wt %, for example at least about 5, 6, 7, 8, 9, 10, 15, 20, 25, 30, 35, 40, 45, 50, 55, or 60 wt % and/or up to about 20, 30, 40, 45, 50, 55, 60, 65, 70, 75, 80, 85, 90, 91, 92, 93, 94, or 95 wt %, based on the total combined weight of the hydrogen peroxide releasing compound and the polymer. For example, the polymer can be present in an amount of about 5 wt % to about 90 wt %, about 10 wt % to about 90 wt %, about 10 wt % to about 80 wt %, about 15 wt % to about 80 wt %, about 15 wt % to about 75 wt %, about 20 wt % to about 75 wt %, about 20 wt % to about 70 wt %, about 25 wt % to about 70 wt %, about 25 wt % to about 65 wt %, about 30 wt % to about 60 wt %, about 35 wt % to about 60 wt %, about 35 wt % to about 55 wt %, about 40 wt % to about 55 wt %, or about 40 wt % to about 50 wt %, based on the total combined weight of the hydrogen peroxide releasing compound and the polymer. In embodiments, the polymer is present in the mixture in an amount of about 90 wt %, based on the total weight of the hydrogen peroxide releasing compound and the polymer. In embodiments, the polymer is present in the mixture in an amount of about 80 wt %, based on the total weight of the hydrogen peroxide releasing compound and the polymer. In embodiments, the polymer is present in the mixture in an amount of about 60 wt %, based on the total weight of the hydrogen peroxide releasing compound and the polymer. In embodiments, the polymer is present in the mixture in an amount of about 50 wt %, based on the total weight of the hydrogen peroxide releasing compound and the polymer. In embodiments, the polymer is present in the mixture in an amount of about 40 wt %, based on the total weight of the hydrogen peroxide releasing compound and the polymer. In embodiments, the polymer is present in the mixture in an amount of about 14 wt %, based on the total weight of the hydrogen peroxide releasing compound and the polymer. In embodiments, the polymer is present in the mixture in an amount of about 8 wt %, based on the total weight of the hydrogen peroxide releasing compound and the polymer.

The polymer can be present in the mixture in an amount of about 1% (w/v) to about 10% (w/v), for example at least about 1, 2, 3, 4, 5, 6, 7, or 8% (w/v) and/or up to about 3, 4, 5, 6, 7, 8, 9, or 10% (w/v), such as about 1% (w/v) to about 9% (w/v), about 2% (w/v) to about 8% (w/v) about 3% (w/v) to about 7% (w/v), about 4% (w/v) to about 6% (w/v), about about 1% (w/v) to about 8% (w/v), about 1% (w/v) to about 5% (w/v), about 2% (w/v) to about 6% (w/v), or about 5% (w/v). In embodiments, the polymer is present in the mixture in an amount of about 1% (w/v). In embodiments, the polymer is present in the mixture in an amount of about 2% (w/v). In embodiments, the polymer is present in the mixture in an amount of about 3% (w/v). In embodiments, the polymer is present in the mixture in an amount of about 4% (w/v). In embodiments, the polymer is present in the mixture in an amount of about 5% (w/v). In embodiments, the polymer is present in the mixture in an amount of about 6% (w/v). In embodiments, the polymer is present in the mixture in an amount of about 7% (w/v). In embodiments, the polymer is present in the mixture in an amount of about 8% (w/v). In embodiments, the polymer is present in the mixture in an amount of about 9% (w/v). In embodiments, the polymer is present in the mixture in an amount of about 10% (w/v).

The hydrogen peroxide releasing compound and the polymer can be present in the mixture in a weight ratio of about 1:19 to about 19:1, for example at least about 1:19, 1:18, 1:15, 1:11, 1:10, 1:9, 1:7, 1:5, 1:4, 1:2, 2:3, or 1:1, and/or up to about 1:5, 1:2, 1:1, 2:1, 3:2, 5:1, 6:1, 8:1, 10:1, 11:1, 15:1, 17:1, or 19:1. For example, the hydrogen peroxide releasing compound and the polymer can be present in the mixture in a weight ratio of about 1:10 to about 15:1, about 1:9 to about 11:1, about 1:4 to about 6:1, about 2:3 to about 3:2, or about 1:1. In embodiments, hydrogen peroxide releasing compound and the polymer can be present in the mixture in a weight ratio of about 1:9. In embodiments, hydrogen peroxide releasing compound and the polymer can be present in the mixture in a weight ratio of about 1:4. In embodiments, hydrogen peroxide releasing compound and the polymer can be present in the mixture in a weight ratio of about 2:3. In embodiments, hydrogen peroxide releasing compound and the polymer can be present in the mixture in a weight ratio of about 1:1. In embodiments, hydrogen peroxide releasing compound and the polymer can be present in the mixture in a weight ratio of about 3:2. In embodiments, hydrogen peroxide releasing compound and the polymer can be present in the mixture in a weight ratio of about 6:1. In embodiments, hydrogen peroxide releasing compound and the polymer can be present in the mixture in a weight ratio of about 11:1.

The mixture can further include a solvent. The solvent can be included for the polymer, the hydrogen peroxide releasing compound, or both. That is, the solvent can be provided to dissolve the polymer, alone, the hydrogen peroxide releasing compound, alone, or both of the polymer and the hydrogen peroxide releasing compound. In embodiments, the mixture is a solution, wherein each of the polymer and the hydrogen peroxide releasing compound are dissolved in the solvent. In embodiments, the polymer is dissolved in the solvent to provide a polymeric solution, and the hydrogen peroxide releasing compound is dispersed in the polymeric solution. In embodiments, the hydrogen peroxide releasing compound is dissolved in the solvent to provide a solution, and the polymer is dispersed within (e.g., throughout) the solution.

Where the polymer is dissolved in the solvent to provide a polymeric solution and the hydrogen peroxide releasing compound is dispersed therein, the mixture should be sufficiently mixed in order to provide a homogenous mixture (e.g., where the distances between each of the hydrogen peroxide releasing compounds in the mixture vary by no more than about 10%). For example, when provided as a coating, the mixture should be homogenous when contacted with the substrate, such that an even and consistent coating is applied. Homogeneity of the coating can be achieved, for example, shaking the coating mixture prior to application. In embodiments where each of the hydrogen peroxide releasing compound and the polymer are fully dissolved in the solvent, then less mixing (e.g., via shaking) prior to application may be needed. For example, where each of the polymer and the hydrogen peroxide releasing compound are dissolved in the solvent, homogeneity of the components is expected.

Where the polymer matrix is provided as a particle, the mixtures disclosed herein can be prepared and processed into particles using microfluidic processes. For example, the particles can be prepared using a T-junction microfluidic chip. The resulting particles can be weaved into, mechanically entangled with, embedded in, encapsulated or contained in, or otherwise in contact with the substrate, as described herein.

The solvent can be an organic solvent. The organic solvent can be selected depending on the particular polymer and the hydrogen peroxide releasing compound. For example, the organic solvent can be selected to dissolve the polymer but not the hydrogen peroxide releasing compound; to dissolve the hydrogen peroxide releasing compound but not the polymer, or to dissolve both of the hydrogen peroxide releasing compound and the polymer. Thus, suitable organic solvents can be chlorinated or non-chlorinated, polar or nonpolar, or protic or aprotic solvents. Examples of suitable organic solvents include, but are not limited to, dichloromethane, acetonitrile, ethanol, methanol, butanol, propanol, and acetone. The solvent can include a single solvent, or a mixture of two or more solvents. In embodiments, the solvent includes dichloromethane, acetonitrile, or both.

The solvent can be included in an amount of about 50% (w/v) to about 98% (w/v), for example at least about 50, 55, 60, 65, 70, 75, 80, 85, 87, or 90% (w/v) and/or up to about 80, 85, 87, 90, 93, 95, 96, 97, or 98% (w/v). For example, the solvent can be present in the mixture in an amount of about 50% (w/v) to about 98% (w/v), about 55% (w/v) to about 98% (w/v), about 60% (w/v) to about 98% (w/v), about 70% (w/v) to about 98% (w/v), about 80% (w/v) to about 98% (w/v), about 85% (w/v) to about 98% (w/v), about 87% (w/v) to about 98% (w/v), about 90% (w/v) to about 98% (w/v), about 93% (w/v) to about 98% (w/v), or about 95% (w/v) to about 98% (w/v). In embodiments, the solvent is present in the mixture in an amount of about 87% (w/v). In embodiments, the solvent is present in the mixture in an amount of about 90% (w/v). In embodiments, the solvent is present in the mixture in an amount of about 93% (w/v). In embodiments, the solvent is present in the mixture in an amount of about 98% (w/v).

In embodiments, the hydrogen peroxide releasing compound includes UHP, the polymer includes PCL, and the solvent includes dichloromethane. In embodiments, the UHP can be present in the mixture in an amount of about 12% (w/v), and the PCL can be present in the mixture in an amount of about 1% (w/v). In embodiments, the UHP can be present in the mixture in an amount of about 6% (w/v), and the PCL can be present in the mixture in an amount of about 1% (w/v). In embodiments, the UHP can be present in the mixture in an amount of about 1% (w/v), and the PCL can be present in the mixture in an amount of about 1% (w/v). In embodiments, the UHP can be present in the mixture in an amount of about 6% (w/v), and the PCL can be present in the mixture in an amount of about 4% (w/v). In embodiments, the UHP can be present in the mixture in an amount of about 5% (w/v), and the PCL can be present in the mixture in an amount of about 5% (w/v). In embodiments, the UHP can be present in the mixture in an amount of about 4% (w/v), and the PCL can be present in the mixture in an amount of about 6% (w/v). In embodiments, the UHP can be present in the mixture in an amount of about 2% (w/v), and the PCL can be present in the mixture in an amount of about 8% (w/v). In embodiments, the UHP can be present in the mixture in an amount of about 1% (w/v), and the PCL can be present in the mixture in an amount of about 9% (w/v). The solvent including dichloromethane can be present in amount to make up the difference remaining after accounting for the polymer and the hydrogen peroxide releasing compound (e.g., to a total of 100% (w/v).

In embodiments, the hydrogen peroxide releasing compound includes UHP, the polymer includes PLGA, and the solvent includes acetonitrile. In embodiments, the UHP can be present in the mixture in an amount of about 12% (w/v), and the PLGA can be present in the mixture in an amount of about 1% (w/v). In embodiments, the UHP can be present in the mixture in an amount of about 6% (w/v), and the PLGA can be present in the mixture in an amount of about 1% (w/v). In embodiments, the UHP can be present in the mixture in an amount of about 1% (w/v), and the PLGA can be present in the mixture in an amount of about 1% (w/v). In embodiments, the UHP can be present in the mixture in an amount of about 6% (w/v), and the PLGA can be present in the mixture in an amount of about 4% (w/v). In embodiments, the UHP can be present in the mixture in an amount of about 5% (w/v), and the PLGA can be present in the mixture in an amount of about 5% (w/v). In embodiments, the UHP can be present in the mixture in an amount of about 4% (w/v), and the PLGA can be present in the mixture in an amount of about 6% (w/v). In embodiments, the UHP can be present in the mixture in an amount of about 2% (w/v), and the PLGA can be present in the mixture in an amount of about 8% (w/v). In embodiments, the UHP can be present in the mixture in an amount of about 1% (w/v), and the PLGA can be present in the mixture in an amount of about 9% (w/v). The solvent including acetonitrile can be present in amount to make up the difference remaining after accounting for the polymer and the hydrogen peroxide releasing compound (e.g., to a total of 100% (w/v).

The mixture can be free of any components other than the polymer, the hydrogen peroxide releasing compound, and the solvent. As used herein, “free of any components other than the polymer, the hydrogen peroxide releasing compound, and the solvent” means that the mixture does not include any other components (e.g., additives, catalysts, dispersants, etc.) such that at least 90 wt % or at least 90% (w/v) of the mixture is attributable to the polymer, hydrogen peroxide releasing compound, and the solvent. In embodiments, at least 90, 95, 97, 98, 99, 99.5, 99.9, or 100 wt %, or at least 90, 95, 97, 98, 99, 99.5, 99.9, or 100% (w/v) of the mixture is attributable to the polymer, the hydrogen peroxide releasing compound, and the solvent.

The mixture can be free of one or more of water, ferric ion, and a peroxidase. As used herein, the term “free of one or more of water, ferric ion, and a peroxidase,” means that the mixture can be free of water, ferric ion, and/or peroxidase, such that each and all of these components are present in a total amount of less than 5 wt %, based on the total weight of the mixture. In embodiments, each and all of water, ferric ion, and/or peroxidase are present in a total amount of less than 5 wt %, 4 wt %, 3 wt %, 2 wt %, 1 wt %, 0.5 wt %, 0.1 wt %, 0.05 wt %, or 0.01 wt %, based on the total weight of the mixture. Additionally, the term “free of one or more of water, ferric ion, and a peroxidase,” means that the mixture can be free of any water, ferric ion, and/or peroxidase, such that each and all of these components are present in the mixture in a total amount of less than 5% (w/v). In embodiments, each and all of water, ferric ion, and/or peroxidase are present in a total amount of less than 5, 4, 3, 2, 1, 0.5, 0.1, 0.05, or 0.01% (w/v). Significantly, as each of water, ferric ion, and peroxidases are capable of dissociating or breaking down the hydrogen peroxide releasing compound to water and oxygen, it is preferred that the presence of these compounds in the mixture is minimized. Nonetheless, with respect to water, it is possible that the hydrogen peroxide releasing compound, such as UHP, even if initially dissolved in water, can be recovered via recrystallization and used in the articles of the disclosure. Alternatively, in embodiments, inclusion of small amounts (e.g., about 0.01 μg/cm² to about 3 μg/cm²) of metal ions, such as ferrous ion, cobalt ion, etc., can be incorporated in the inner layers of porous substrate, such as a face mask, to help facilitate the dissociation and break down of excess hydrogen peroxide (e.g., released from the hydrogen peroxide releasing agent) to oxygen and water. In such embodiments, the metal ions should not be placed in the same layer as the polymer matrix of the disclosure. Such embodiments can be advantageous where the hydrogen peroxide releasing compound has been dissociated into hydrogen peroxide and water, and the hydrogen peroxide is released in excessive amounts. Where the article is a face mask, incorporation of these small amounts of metal ions can advantageously break down the trapped hydrogen peroxide to reduce toxicity concerns and to comply with OSHA's standard of less than 1 ppm of hydrogen peroxide in breathing air. Additionally, where the article is a face mask, the metal ions should be placed in a layer closer to the mouth than the polymer matrix, so as to facilitate the dissociation of hydrogen peroxide prior to reaching the user's mouth.

The methods of the disclosure can include drying, curing, or otherwise solidifying the article. The article can be dried at room temperature (e.g., under ambient conditions), freeze-dried, or dried in a heated oven (e.g., at a temperature of about 40, 50, 60, 70, 80, 90, or 100° C.). Throughout the drying process, the solvent present in the mixture can be evaporated such that the article, including the polymer matrix, as described herein, can be provided on or in contact with the substrate.

### Kits for Preparing Articles

The disclosure additionally provides kits for preparing the articles of the disclosure. The kits can include the mixture, as described herein, including the polymer, the hydrogen peroxide releasing compound, and, optionally, the solvent. Alternatively, the kits can include a first mixture including the polymer and the hydrogen peroxide releasing compound, and a second mixture including the solvent. Alternatively, the kits can include a first mixture including the polymer, a second mixture including the hydrogen peroxide releasing compound, and a third mixture including the solvent. The mixture or mixtures can be packaged in a bottle (e.g., a spray bottle), can (e.g., an aerosol can), a drum, or pallet within the kit. The kits can further include instructions to contact a substrate with the mixture as described herein (e.g., dipping, brushing, melt-spinning, extruding, molding, spraying, weaving, entangling, embedding, etc.) to provide a coated substrate. The kits can further include instructions to mix the mixture (e.g., via shaking, stirring, or otherwise agitating) immediately prior to contacting the substrate. Where the components (e.g., the polymer, the hydrogen peroxide releasing compound, and/or the solvent) are packaged separately, the kits can include instructions to mix each of the mixtures together immediately prior to contacting the substrate. The kits can further include instructions to dry the article after contacting as described herein (e.g., at room temperature, via freeze-drying, or in a heated oven).

The above-described aspects and embodiments can be better understood in light of the following examples, which are merely intended to be illustrative and are not meant to limit the scope in any way.

### EXAMPLES

### Example 1: Preparation and Evaluation of Filters with UHP and PCL

A nonwoven polymer fabric made from polypropylene was obtained from commercially available N95 face masks. A fine powder of UHP (having a mesh of 300 or higher) was dispersed in a polymeric solution prepared by dissolving 1% (w/v) PCL in dichloromethane (DCM), to provide a mixture. The UHP was provided in the mixtures in concentrations of 12% (w/v), 6% (w/v), 3% (w/v), 1% (w/v), and 0.1% (w/v). The nonwoven fabrics were immersed in the mixtures for few seconds (approximately 5 seconds) and were removed and left to dry at room temperature. To ensure homogenous distribution of mixtures throughout the fabric, the fabric was rotated until the solvent was evaporated. The coated fabrics were left to dry further overnight.

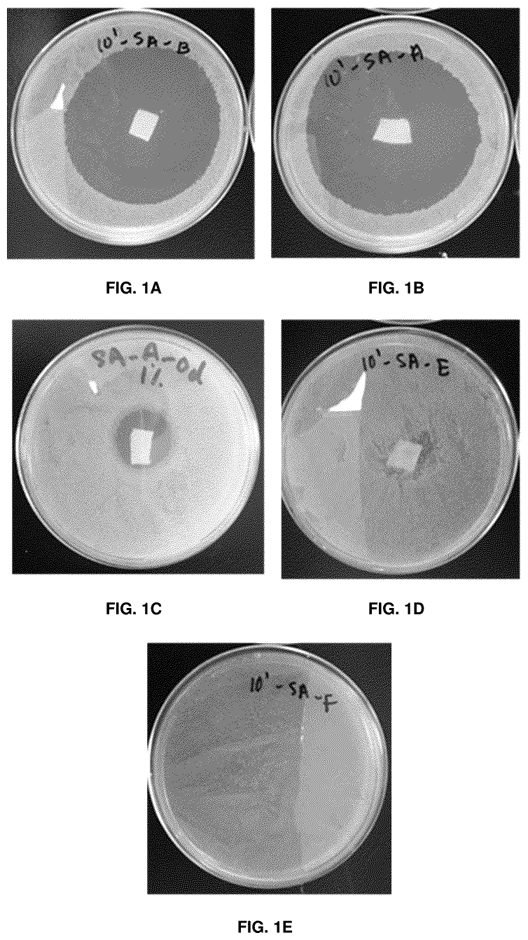

The antimicrobial properties of the coated fabrics were evaluated. To observe these properties, an antibiogram disc assay was used, where the coated fabrics were placed on the surface of agarose hydrogel. This assay allowed for the evaluation of the diffusion of hydrogen peroxide into the agarose gel and inhibition of bacteria growth. In brief, squares of the coated fabric were cut and exposed to pre-cultured E. coli and S. Aureus nutrient-agar dishes (0.1 mL of 106 colony-forming units (CFU)/mL per Petri dish). After incubation at 37° C. for 24 hours, the inhibition zone around the fabric was recorded to provide a quantitative evaluation of the antibacterial activities of the fabrics. The antibacterial activities were compared to a Control (i.e., Control 1, a fabric sample immersed in a mixture of PCL in DCM only) and a Blank (i.e., an empty dish). Images of the antibacterial activity of each of the fabrics prepared from the 12% (w/v), 6% (w/v), and 1% (w/v) UHP mixtures, as well as the control and the blank, are provided in FIGS. 1A-1E. These results demonstrate that the PCL, alone, did not impart any significant antibacterial properties. The results from each of the tested samples are provided in Table 1, below.

TABLE 1
Composition of Mixtures used to Coat Fabric
and Initial Antibacterial Activity

| | | | | Antibacterial Activity |
|---|---|---|---|---|
| | | | | (diameter of |
| Sample | UHP* | PCL* | DCM* | inhibition zone, mm) |
| 1 | 12 | 1 | 87 | 95 |
| 2 | 6 | 1 | 93 | 51 |
| 3 | 3 | 1 | 96 | 36 |
| 4 | 1 | 1 | 98 | 24 |
| 5 | 0.1 | 1 | 99.9 | 0 |
| Control 1 | 0 | 1 | 99 | 0 |
| Blank | 0 | 0 | 0 | — |

*Amounts provided in % (w/v)

Upon drying, the coating of Sample 1 included about 92.3 wt % UHP and 7.7 wt % PCL; Sample 2 included about 85.7 wt % UHP and about 14.3 wt % PCL; Sample 3 included about 75 wt % UHP and about 25 wt % PCL; Sample 4 included about 50 wt % UHP and 50 wt % PCL; and Sample 5 included about 9.1 wt % UHP and about 90.9 wt % PCL. The coating of the Control 1 included 100 wt % PCL.

To determine the bactericidal rate of coated fabrics, S. aureus was used as representative gram-positive bacteria. Briefly, bacteria suspensions of 103 CFU/mL were prepared and spread onto the coated fabrics (1 mL/10 cm²). Different contact times (20 min, 60 min, 120 min) were tested. At the end of the prescribed contact time, the inoculated coated fabrics were mixed with 10 mL PBS solution. The bacteria-containing supernatant was serially diluted and cultured in nutrient agar dishes. The number of colonies were counted 24 hours after incubation at 37° C. The reduction percentile of each sample was calculated relative to the control (i.e., having no sample), and is provided in Table 2, below.

TABLE 2
Reduction Percentile of Coated Fabrics after Contamination
and Incubation Relative to Control

| Sample | 20 Minutes | 60 Minutes | 120 minutes |
|---|---|---|---|
| 1 | 99% | 100% | 100% |
| 2 | 95% | 99% | 100% |
| 4 | 90% | 95% | 99% |
| Control 1 | 0% | 0% | 0% |

As shown by these data, the articles of the disclosure were effective in killing S. aureus after 120 minutes of exposure. As the amount of UHP increased relative to the PCL, the bacteria were deactivated more quickly, as shown by Samples 1 and 2, deactivating the bacteria within 20 minutes and 60 minutes, respectively.

Example 1 therefore demonstrates that the articles of the disclosure, including the polymer matrix of the disclosure provided as a coating (e.g., via dip coating on the substrate) demonstrate antibacterial and bactericidal effects.

### Example 2: Comparison of UHP and Calcium Peroxide

Using the methods described in Example 1, two coating mixtures including PCL/Calcium Peroxide (PCL/CPO) and PCL/UHP were prepared. The ratios of polymer:peroxide in the final coating were adjusted to 1:6, 1:2 and 1:1 for each mixture. The antimicrobial properties were evaluated as described in Example 1 and the inhibition zone around coated pieces of fabric (1 cm×1 cm) were measured. In particular, the coated fabrics were placed in an incubator having a relative humidity (RH) of 95-100%, a CO₂ concentration of 5% and a temperature of 37° C. This environment was selected to emulate the exhaled human breath that has around 95% humidity, 4-5% CO₂, and ˜37° C. in order to assess the stability of coated fabrics under such conditions. After incubation times of up to 8 days, the antimicrobial properties of the samples, using disc antibiogram assay as described above, were evaluated.

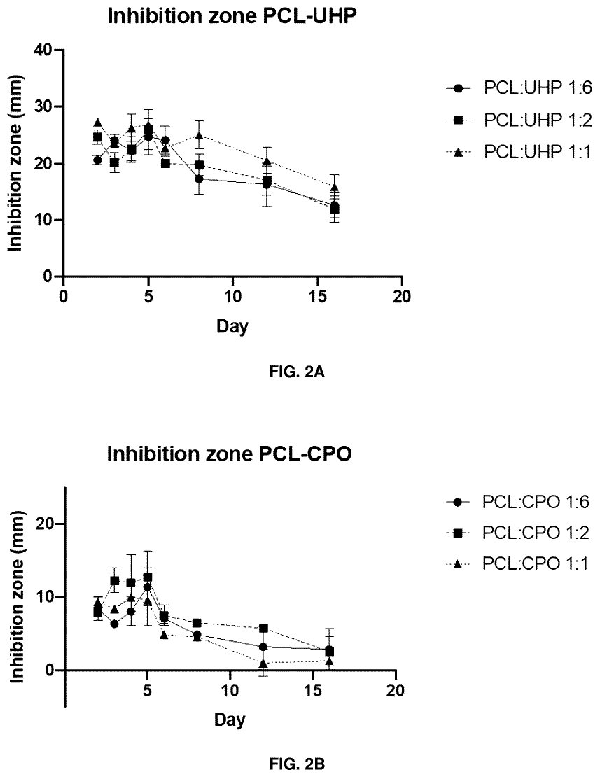

Results of these measurements are shown in FIG. 2A (PCL/UHP) and FIG. 2B (PCL/CPO). It was observed that the coated fabrics exhibited antimicrobial activity over eight days of measurement as shown. The data also showed that UHP released higher amounts of hydrogen peroxide, creating a larger inhibition zone around the fabric.

Accordingly, Example 2 demonstrates that each of UHP, as well as CPO, as hydrogen peroxide releasing agents in the articles of the disclosure demonstrate antibacterial effects.

### Example 3: Preparation and Evaluation of Filters with UHP and PLGA

As in Example 1, a nonwoven polymer fabric made from polypropylene was obtained from commercially available N95 face masks. A fine powder of UHP (having a mesh of 300 or higher) was dispersed in a polymeric solution prepared by dissolving PLGA in acetonitrile (ACN), to provide a mixture. The nonwoven fabrics were immersed in the mixtures for few seconds and were removed and left to dry at room temperature. To ensure homogenous distribution of mixtures throughout the fabric, the fabrics were rotated until the solvent was evaporated. The coated fabrics were left to dry further overnight. The tested mixtures are provided in Table 3, below.

TABLE 3
Composition of Mixtures used to Coat Fabrics

| | | | | Antibacterial Activity |
|---|---|---|---|---|
| | | | | (diameter of |
| Sample | UHP* | PLGA* | ACN* | inhibition zone, mm) |
| 6 | 4 | 6 | 90 | 25 |
| 7 | 2 | 8 | 90 | 5 |
| 8 | 1 | 9 | 90 | 2 |

*Amounts provided in % (w/v)

Upon drying, the coating of Sample 6 included about 40 wt % UHP and 60 wt % PLGA; Sample 7 included about 20 wt % UHP and about 80 wt % PLGA; and Sample 8 included about 10 wt % UHP and about 90 wt % PLGA.

The antimicrobial activity of the coated fabrics against S. aureus was evaluated and confirmed using disc antibiogram assay as described, and as shown in Table 3. Formation of inhibition zone around all samples confirm that this preparation approach was suitable for preparation of the articles of the disclosure.

### Example 4: Preparation of Portable Bag

To explore a suitable application of the articles of the disclosure, bags including UHP/PCL coated lining, as well as gas permeable bags containing UHP particles were prepared.

In particular, a lining made of nonwoven polypropylene (PP) coated with UHP/PCL in a 6:1 ratio was inserted into a small plastic zip bag (12 cm×12 cm) (“Bag 1”). The coated fabric was covered with another layer of nonwoven PP with higher density to avoid direct contact of items within the bag to the UHP/PCL coated fabric. Additionally, a small sachet made of high-density nonwoven PP was filled with 2 g of UHP particles (as powder) and sealed. The sachet was placed into a small plastic zip bag, similar to that of Bag 1 (“Bag 2”). As a control, a similar bag without lining was used (“Control Bag”).

It was observed that the hydrogen peroxide gas concentration exceeded 100 ppm within 10 minutes inside the bag as measured by a commercially available hydrogen peroxide indicator (Bartovation, US). Prior studies have shown that similar concentrations of vaporized hydrogen peroxide can redicate C. difficile spores within 20 min.

To evaluate the antimicrobial efficacy, a piece of nonwoven PP (1 cm×3 cm) was inoculated with 10⁸ S. Aureus per square centimeter and placed into each of Bag 1 (having the coated lining), Bag 2 (containing the filled sachet), and Control Bag. All bags were place at room temperature for 12 hours and the bacterial loading of contaminated nonwoven items were measured as described. Each of Bag 1 and Bag 2 eradicated 100% of bacteria. In contrast, the Control Bag showed the recovery of 10⁸ bacteria/cm², indicating 0% efficiency.

Thus, Example 4 demonstrates that a lining or a sachet prepared in accordance with the embodiments of the disclosure are capable of providing hydrogen peroxide gas in small amounts to an interior environment of a bag or container. As the hydrogen peroxide gas accumulated in the interior space of the bag, it was able to disinfect and/or sterilize items within the bag without directly contacting them.

### Example 5: Preparation of CPO/PLGA Particles

### Fabrication of a T-Junction Microfluidic Chip

Microfluidic particle generating T-junction microfluidic chips were created using soft lithography techniques. Specifically, negative photoresist SU-8 2125 was spin coated on silicon wafers to create a 200 micron thick layer according to the manufacturer's data sheet (500 rpm, 10s, 100 rpm/s², 3000 rpm, 30 s, 300 rpm/s²), and baked on a hot plate at 65° C. for 5 min and another 30 min at 95° C. for soft bake. The wafers were then exposed to UV light (365 nm, 300 mJ/cm²) using a Karl Suss MA6 mask aligner with hard contact, and post-exposure baked at 65° C. for 5 min and another 15 min at 95° C. PDMS base and curing agent were mixed in 10:1 ratio, and then poured onto the silicon wafer. The silicon wafer with PDMS mold was placed into a vacuum chamber to remove the bubbles in PDMS for 1 hr, and subsequently placed in an oven at 90° C. for 2 hrs. After curing, the PDMS layer was peeled from the silicon wafer. Inlets and outlets to the microfluidic chip were made using 23G blunt needles. The PDMS samples were then treated with air plasma (400 mTorr) (PE-25, Plasma Etch) for 2 minutes and then bonded to a glass slide for permanent bond (34, 35).

### Preparation of Microspheres by the T-Junction Microfluidic Chip

The T-junction microfluidic devices were utilized to generate CPO/PLGA microdroplets. To prepare a solution of PLGA and CPO in DCM, CPO stock solution was first prepared by mixing CPO into a DCM solvent with 5% (v/v) triethoxyvinylsilane at 100 mg/ml and was subjected to sonication in an ice/water bath (Misonix sonicator S-4000—Boston Laboratory Equipment, amplitude set at 30%) for 30 mins to breakdown CPO aggregates for homogeneous CPO dispersion and to prevent the clogging of microfluidic chips. An appropriate amount of PLGA powder was added subsequently to the DCM solution.

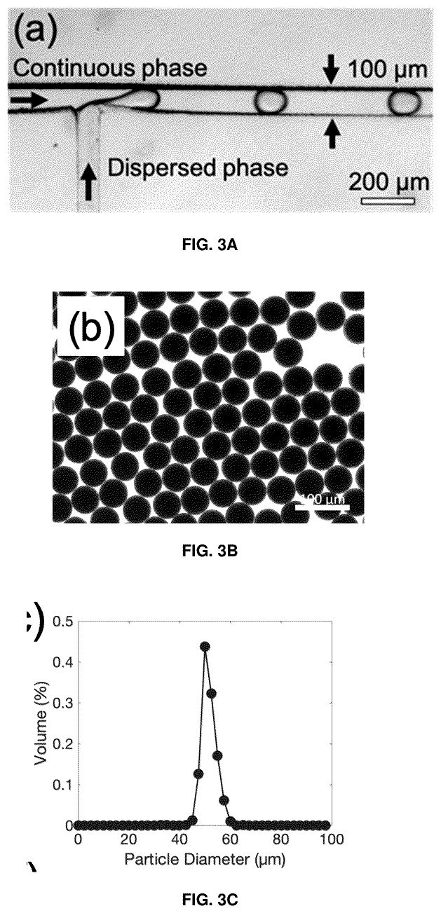

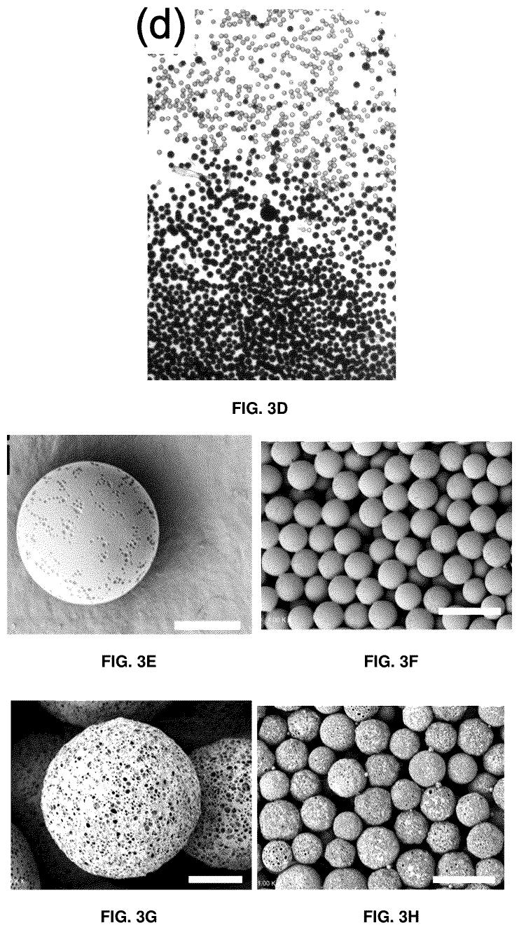

A solution of PLGA and CPO was introduced into dichloromethane (DCM) as a disperse phase, and water solution containing 1% (w/v) polyvinyl alcohol (PVA) was introduced as a continuous phase (FIG. 3A). The flow rates of disperse phase and continuous phase were controlled by syringe pumps (model NE-1010, New Era Pump Systems, Inc., Wantagh, NY) and kept at 3 and 1 ml/hr, respectively.

PLGA/CPO microdroplets generated from the microfluidic chips were collected in a glass Petri dish for DCM to evaporate. To increase the evaporation rate, the Petri dish was mounted onto a shaker (50 rpm, Fisherbrand™ wave motion shaker, Fisher Scientific Co.). After the evaporation of DCM, solid microparticles (MPS) were formed. Subsequently, a negative pressure (˜50 kPa) was applied for 10 mins to remove the remaining DCM. The MPS were then centrifuged (4000 rpm, 3 mins), and washed with excess DI water to remove residual PVA, and were lyophilized overnight into dried powder for the following experiments.

The MPS were analyzed using ImageJ software (FIG. 3B). The average diameter of MPS was found to be 53.0±3.0 μm, with a narrow size distribution (FIG. 3C). The diameter of MPS can be tuned by adjusting the flow rates and the concentration of PLGA and CPO in the dispersed phase.

FIG. 3D shows the optical images of PLGA MPS laden with (lower) and without (upper) CPO (MPS generated from 1.5% CPO and 3.5% PLGA (w/v) in dispersed phase). The darker color of the CPO laden MPS implies successful encapsulation of CPO (opaque) inside the particles. FIGS. 3E-H shows the scanning electron microscopy micrographs of PLGA MPS without (3E, 3F) and with (3G, 3H) CPO (where CPO loading was approximately 15 wt %). It was also observed that CPO loading resulted in a porous particle surface. Thus, CPO was believed to act as a porogen, leaving pores after interacting with water.

The loading efficiency of CPO in PLGA MPS was also investigated, and the amounts of CPO encapsulated within MPS were characterized by inductively coupled plasma mass spectrometry (ICP-MS). The CPO concentration within 5% (w/v) of PLGA solution in DCM was adjusted from 0 to 2% (w/v). After ICP-MS characterization, it was found that the CPO mass loading inside PLGA MPS was linear with the CPO amount of about 56% of the CPO concentration in the dispersed phase This indicated that 44% of the CPO was lost or unaccounted for during the MPS generation process. This loading efficiency could be due to CPO reacting with water on the microdroplet surface and/or precipitation of CPO inside the MPS generation system, including syringes, needles, and tubing. In addition, it was found that the MPS generated by a dispersed solution having CPO mass percentage exceeding 40%, for example, 2.5% (w/v) CPO and 2.5% (w/v) PLGA (corresponding CPO mass percentage is 50 wt %) were fragile and ended up breaking in one of the later processes. Accordingly, the CPO concentration was maintained at less than 2.5% (w/v) in the dispersed solution to create robust particles.

Measurement of Hydrogen Peroxide Release Kinetics from Microspheres

To demonstrate sustained peroxide release kinetics for two weeks, several parameters related to release kinetics from CPO laden MPS were investigated, including MPS diameter, CPO loading, and MPS concentrations in PBS.

The hydrogen peroxide release kinetics of the CPO/PLGA MPS were determined indirectly by measuring the dissolved oxygen levels in phosphate buffered saline (PBS) after breaking down the hydrogen peroxide with catalase. Dissolved oxygen levels were measured by an oxygen sensor probe (ISO-OXY-2, World Precision Instruments), which was connected to the free radical analyzer (TBR1025, World Precision Instruments). MPS with different CPO loading and MPS with different dimensions were evaluated. The MPS were added into 1 ml of PBS with 100 U/ml catalase and placed into individual glass vials, and PBS was purged with N₂ for 10 mins to create a hypoxic solution (1% O₂). Catalase was used to catalyze the decomposition of hydrogen peroxide to water and oxygen. All glass vials were placed in a modular incubator hypoxia chamber (MIC-101, Billups-Rothenberg) and then flushed with a customized gas mixture (1% O₂, 5% CO_{2,} and 94% N2). After fully flushed, the hypoxic chamber with glass vials containing samples was sealed and placed into a cell culture incubator at 37° C.

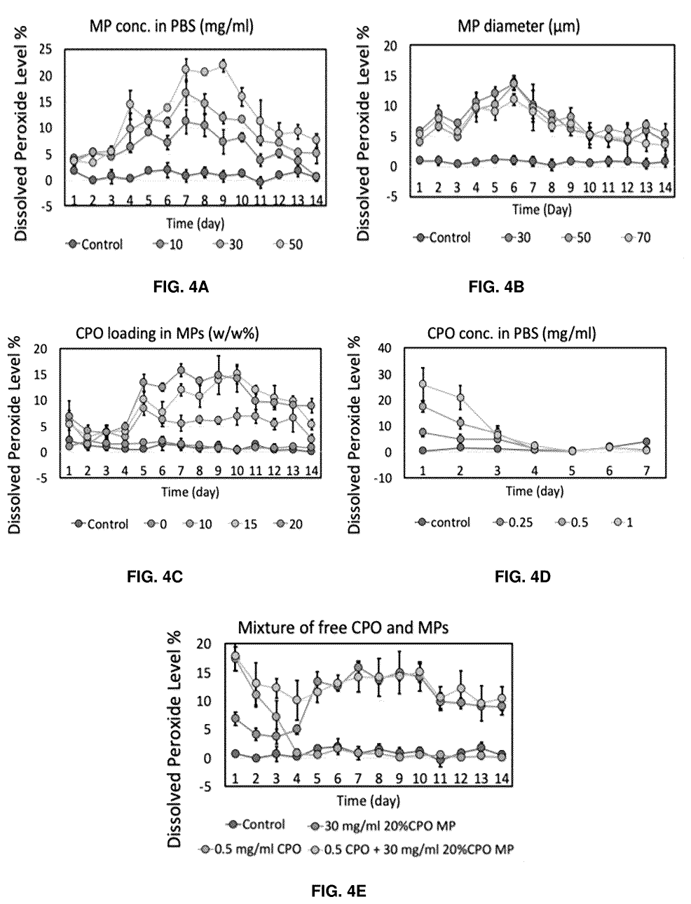

First, the effect of the MPS concentration on the peroxide levels in PBS was investigated (FIG. 4A). The MPS diameter and CPO loading in MPS were set to 50 μm and 15% (w/v), respectively. As expected, the dissolved peroxide levels in the PBS solution of the control samples (PBS without any CPO/PLGA MPS) remained at ˜0% level throughout the entire experiment. This confirmed that oxygen did not diffuse into the chamber from the outside and as such the hypoxic conditions were maintained during the course of the experiment. For the release profiles of CPO/PLGA MPS with three concentrations (10, 30, and 50 mg/ml), the dissolved oxygen levels started around 5% at day 1; showed a relatively significant increase until day 4; reached a peak which occurred around days 6 to 9; and then slightly decreased during the following days. Comparing the results obtained from three different MPS concentrations, it was observed that all of the oxygen releasing kinetic profiles had a similar trend and the amount of released oxygen correlated well with the MPS concentrations in PBS, indicating that the hydrogen peroxide releasing kinetics was independent of the MPS concentration in the range from 10 to 50 mg/ml. Accordingly, an MPS concentration of 30 mg/ml was selected for the remainder of the experiments.

Second, the influence of MPS diameter to oxygen release kinetics was investigated. CPO/PLGA MPS that were 30, 50, and 70 μm in diameter were prepared (MPS concentration and CPO loading were set to 30 mg/ml and 15%, respectively) and the results are shown in FIG. 4B. Surprisingly and advantageously, the change in MPS diameter in this range showed no significant difference in hydrogen peroxide release kinetics. Without intending to be bound by theory, it was believed this was due to the porous structure of the MPS.

Third, the CPO loading in the CPO/PLGA MPS was varied from 10 wt % to 20 wt % to investigate the influence of CPO loading in peroxide release kinetics. Significant peroxide release occurred started around day 4 and lasted until day 14, as shown for all samples in FIG. 4C. These data showed that higher MPS concentrations led to increased peroxide levels. Similarly, increasing the CPO amount in the MPS also lead to increased peroxide levels as expected. MPS with 20% CPO loading were chosen because they released the most amount of peroxide without disintegration of particles as noted at higher loading densities.

Next, free CPO was to the PBS solution at different concentrations and the effect on the peroxide release kinetics was observed. Elevated peroxide levels were obtained in the early stages (0-3 days) of the experiment where the levels decreased gradually (FIG. 4D). A concentration of 0.5 mg/ml free CPO was chosen because it was found that it had the ability to provide suitable dissolved peroxide levels. Free CPO was also mixed with CPO/PLGA MPS with the goal to provide a flat peroxide release profile during the course of the experiment. The measured peroxide profile is shown in FIG. 4E. The release profile of the mixture of 0.5 mg/ml CPO and 30 mg/ml 20 wt %) CPO/PLGA MPS showed a relatively stable from early day of incubation, which was desirable.

Thus, Example 5 demonstrates the preparation of the polymer matrices of the disclosure as particles, and the efficacy of such particles in releasing hydrogen peroxide.

### Example 6: Evaluation of Antiviral Properties

To evaluate the antiviral activity of the coated filters, spike (SARS-COV-2) pseudo-typed virus (obtained from BPS Bioscience, #79942, San Diego, CA) is used. The antiviral activity is determined according to the AATCC 100 method (Assessment of Antibacterial Finishes on Textile Materials, Modified for Viruses) at BSL2 biosafety level.

Briefly, the stock of the virus is thawed and diluted to 10⁵, 10⁴, and 10³ PFU/mL. The viral inoculum is loaded with organic soil to the appropriate level (5% goat serum). A 0.9 ml inoculum volume is applied to the test samples and Control 1 (having an size of 3 cm×3 cm), ensuring that the inoculum touched only the samples. A 0.9 ml inoculum volume is also be applied to a second control (Control 2, having only the PCL) to serve as a “Time Zero” control. The “Time Zero” control is immediately neutralized in the appropriate media, which is serially diluted and plated in quadruplicate to host cell monolayers (ACE2-HEK293 Recombinant Cell Line, BPS Bioscience, #79951). The test samples and Control 1 are incubated at 37° C. for the duration of 1 min, 10 min and 1 hour. At the end of the prescribed contact time, the test samples and Control 1 are neutralized. The harvest suspensions are serially diluted, and each dilution is plated in quadruplicate to host cell monolayers.

A transduction assay is incubated at 37° C. for the test virus for four days. After transduction, a ONE-Step™ Luciferase reagent is prepared by adding 50 μL of ONE-Step™ Luciferase Assay reagent per well. The samples are incubated at room temperature for about 15 to 30 minutes and the luminescence is measured using a luminometer. The transduction efficacy is determined by measuring the luciferase activity. This assay is performed in an atmosphere containing 90-100% relative humidity and 5% CO₂ inside an incubator at 37° C.

What is claimed is:

1. An article comprising:
a substrate; and,
a polymer matrix comprising a hydrogen peroxide releasing compound dispersed within the polymer matrix;
wherein the polymer matrix is in contact with the substrate.

2. The article of claim 1, wherein the substrate comprises a nonwoven material.

3. The article of claim 1 or 2, wherein the substrate comprises a woven material, a polymeric material, or a metal alloy.

4. The article of claim 2 or 3, wherein the nonwoven material or the woven material comprises polypropylene fiber, polyacrylic acid fiber, polyurethane fiber, polyester fiber, fiberglass fiber, cellulose, hemp, jute, flax, ramie, sisal, bagasse, banana fiber, lacebark, silk, sinew, catgut, wool, sea silk, mohair, angora, cashmere, collagen, actin, nylon, dacron, rayon, bamboo fiber, modal, diacetate fiber, triacetate fiber, copolyester, viscose, polylactide, polyethylene terephthalate, or a combination thereof.

5. The article of claim 3, wherein the polymeric material comprises high-density polyethylene (HDPE) or polyethylene terephthalate (PET).

6. The article of claim 3, wherein the metal alloy comprises steel.

7. The article of any one of the preceding claims, wherein the hydrogen peroxide releasing compound comprises one or more of urea hydrogen peroxide (UHP) and a metal peroxide.

8. The article of claim 7, wherein the metal peroxide comprises one or more of calcium peroxide, magnesium peroxide, silver peroxide, copper peroxide, and zinc peroxide.

9. The article of any one of the preceding claims, wherein the polymer matrix comprises a biodegradable polymer selected from one or more of polyglycolic acid (PGA), polycaprolactone (PCL), polylactic acid (PLA), polyurethane (PU), a polyester, and poly (lactic-co-glycolic acid) (PLGA).

10. The article of any one of the preceding claims, wherein the polymer matrix comprises a nonbiodegradable polymer.

11. The article of any one of the preceding claims, wherein the hydrogen peroxide releasing compound is present in an amount in a range of about 0.1 wt % to about 99.5 wt %, based on the total weight of the polymer matrix.

12. The article of any one of the preceding claims, wherein the hydrogen peroxide releasing compound is present in an amount in a range of about 5 wt % to about 95 wt %, based on the total weight of the polymer matrix.

13. The article of any one of the preceding claims, wherein the polymer is present in an amount in a range of 5 wt % to about 95 wt %, based on the total weight of the polymer matrix.

14. The article of any one of the preceding claims, wherein the hydrogen peroxide releasing compound and the polymer are present in a weight ratio in a range of about 1:19 to about 19:1.

15. The article of claim 14, wherein the weight ratio is in a range of about 1:10 to about 15:1.

16. The article of any one of the preceding claims, wherein the polymer matrix is provided as a coating, a particle, or both.

17. The article of any one of the preceding claims, wherein the article is an antimicrobial article effective against one or more of a fungus, gram-positive bacteria, gram-negative bacteria, spore of bacteria, and a virus.

18. The article of claim 17, wherein the fungus comprises one or more of Candida albicans, Candida auris, Cryptococus gattii, Cryptococcus neoformans, Blastomyces dermatitidis, Paracoccidioides brasiliensis, Paracoccidioides lutzii, Coccidioides immitis, Coccidioides posadasii, Histoplasma capsulatum, Fusarium solani, Fusarium oxysporum, Fusarium verticillidioidis, Fusarium monoliforme, Aspergillus niger, Aspergillus flava, Aspergillus fumigatus, Aspergillis parasiticus, and Pneumocystis jirovecii.

19. The article of claim 17, wherein the gram-negative bacteria comprise one or more of Escherichia coli, Pseudomonas aeruginosa, Klebsiella pneumoniae, Klebsiella oxytoca, Klebsiella terrigena, Klebsiella planticola, Acinetobacter baumannii, Acinetobacter calcoaceticus, and Mycobacterium tuberculosis.

20. The article of claim 17, wherein the gram-positive bacteria comprises Staphylococcus aureus, methicillin-resistant Staphylococcus aureus, Clostridium difficile, and vancomycin-resistant Enterococcus.

21. The article of claim 17, wherein the virus causes a viral disease selected from one or more of COVID-19, chickenpox, influenza, herpes, human immunodeficiency virus (HIV/AIDS), human papillomavirus (HPV), infectious mononucleosis, mumps, measles, rubella, shingles, viral gastroenteritis, viral hepatitis, viral meningitis, and viral pneumonia.

22. The article of any one of the preceding claims, wherein the article is bacteriostatic.

23. The article of any one of the preceding claims, wherein the article is bactericidal.

24. The article of any one of the preceding claims, wherein hydrogen peroxide is released from the hydrogen peroxide releasing compound when the article is exposed to a relative humidity (RH) of at least about 5%.

25. The article of any one of the preceding claims, wherein hydrogen peroxide is released from the hydrogen peroxide releasing compound when the article is exposed to a relative humidity (RH) of about 5% to about 25%.

26. The article of any one of claims 1-24, wherein hydrogen peroxide is released from the hydrogen peroxide releasing compound when the article is exposed to a relative humidity (RH) of at least about 30%.

27. The article of any one of claims 1-24 and 26, wherein hydrogen peroxide is released from the hydrogen peroxide releasing compound when the article is exposed to a relative humidity (RH) of about 90% to about 100%.

28. The article of any one of the preceding claims, wherein the article is incorporated into a filter, a lining material, a medical device, a tool, or a bedding material.

29. The article of claim 28, wherein the bedding material is a mattress, a mattress pad, a sheet, a pillow case, a comforter, or a duvet cover.

30. The article of claim 28, wherein the filter is a face mask, a respirator, an HVAC filter, a vacuum filter, or a HEPA filter.

31. The article of claim 28, wherein the medical device is a disinfectant wipe, a wound dressing, or a biohazard reservoir.

32. The article of claim 28, wherein the lining material is provided in an interior of a bag.

33. The article of claim 28, wherein the tool is a mobile phone, a cosmetic tool, a utensil, or a minor surgical tool.

34. The article of any one of the preceding claims, wherein the hydrogen peroxide releasing compound comprises UHP and the polymer matrix comprises PCL.

35. The article of claim 34, wherein
the UHP is present in an amount of about 92 wt %, based on the total weight of the polymer matrix; and,
the PCL is present in an amount of about 8 wt %, based on the total weight of the polymer matrix.

36. The article of claim 34, wherein
the UHP is present in an amount of about 86 wt %, based on the total weight of the polymer matrix; and,
the PCL is present in an amount of about 14 wt %, based on the total weight of the polymer matrix.

37. The article of claim 34, wherein
the UHP is present in an amount of about 60 wt %, based on the total weight of the polymer matrix; and,
the PCL is present in an amount of about 40 wt %, based on the total weight of the polymer matrix.

38. The article of claim 34, wherein
the UHP is present in an amount of about 50 wt %, based on the total weight of the polymer matrix; and,
the PCL is present in an amount of about 50 wt %, based on the total weight of the polymer matrix.

39. The article of any one claims 1-33, wherein the hydrogen peroxide releasing compound comprises UHP and the polymer matrix comprises PLGA.

40. The article of claim 39, wherein
the UHP is present in an amount of about 40 wt %, based on the total weight of the polymer matrix; and,
the PLGA is present in an amount of about 60 wt %, based on the total weight of the polymer matrix.

41. The article of claim 39, wherein
the UHP is present in an amount of about 20 wt %, based on the total weight of the polymer matrix; and
the PLGA is present in an amount of about 80 wt %, based on the total weight of the polymer matrix.

42. The article of claim 39, wherein
the UHP is present in an amount of about 10 wt %, based on the total weight of the polymer matrix; and
the PLGA is present in an amount of about 90 wt %, based on the total weight of the polymer matrix.

43. The article of any one of claims 1-42, wherein the article is permeable to a gas.

44. A method of forming an article, comprising:
contacting a surface of a substrate with a mixture comprising a hydrogen peroxide releasing compound and a polymer to provide the article.

45. The method of claim 44, wherein contacting comprises dipping, brushing, melt-spinning, extruding, molding, or spraying.

46. The method of claim 44 or 45, wherein the mixture further comprises a solvent for the polymer, the hydrogen peroxide releasing compound, or both.

47. The method of claim 46, wherein the mixture is a solution, wherein each of the polymer and the hydrogen peroxide releasing compound are dissolved in the solvent.

48. The method of claim 46, wherein the polymer is dissolved in the solvent to provide a polymeric solution, and the hydrogen peroxide releasing compound is dispersed in the polymeric solution.

49. The method of any one of claims 46-48, wherein the solvent comprises an organic solvent.

50. The method of claim 49, wherein the organic solvent comprises one or more of dichloromethane, acetonitrile, ethanol, methanol, butanol, propanol, and acetone.

51. The method of any one of claims 44-50, wherein the mixture is free of one or more of water, ferric ion, and a peroxidase.

52. The method of any one of claims 44-51, wherein the hydrogen peroxide releasing compound is present in the mixture in an amount in a range of about 5 wt % to about 95 wt %, based on the total weight of the hydrogen peroxide releasing compound and the polymer.

53. The method of any one of claims 44-52, wherein the hydrogen peroxide releasing compound is present in the mixture in an amount in a range of about 1% (w/v) to about 20% (w/v).

54. The method of any one of claims 44-53, wherein the polymer is present in the mixture in an amount in a range of about 5 wt % to about 95 wt %, based on the total weight of the hydrogen peroxide releasing compound and the polymer.

55. The method of any one of claims 44-54, wherein the polymer is present in the mixture in an amount in a range of about 1% (w/v) to about 10% (w/v).

56. The method of any one of claims 44-55, wherein the hydrogen peroxide releasing compound and the polymer are present in a weight ratio of about 1:10 to about 15:1.

57. The method of any one of claims 46-56, wherein the hydrogen peroxide releasing compound comprises UHP, the polymer comprises PCL, and the solvent comprise dichloromethane.

58. The method of claim 57, wherein
the UHP is present in the mixture in an amount of about 12% (w/v); and,
the PCL is present in the mixture in an amount of about 1% (w/v).

59. The method of claim 57, wherein
the UHP is present in the mixture in an amount of about 6% (w/v); and,
the PCL is present in the mixture in an amount of about 1% (w/v).

60. The method of claim 57, wherein
the UHP is present in the mixture in an amount of about 1% (w/v); and,
the PCL is present in the mixture in an amount of about 1% (w/v).

61. The method of any one of claims 46-56, wherein the hydrogen peroxide releasing compound comprises UHP, the polymer comprises PLGA, and the solvent comprise acetonitrile.

62. The method of claim 61, wherein
the UHP is present in the mixture in an amount of about 6% (w/v); and,
the PLGA is present in the mixture in an amount of about 4% (w/v).

63. The method of claim 61, wherein
the UHP is present in the mixture in an amount of about 5% (w/v); and,
the PLGA is present in the mixture in an amount of about 5% (w/v).

64. The method of claim 61, wherein
the UHP is present in the mixture in an amount of about 4% (w/v); and,
the PLGA is present in the mixture in an amount of about 6% (w/v).

65. The method of claim 61, wherein
the UHP is present in the mixture in an amount of about 2% (w/v); and
the PLGA is present in the mixture in an amount of about 8% (w/v).

66. The method of claim 61, wherein
the UHP is present in the mixture in an amount of about 1% (w/v); and
the PLGA is present in the mixture in an amount of about 9% (w/v).

67. The method of any one of claims 44-66, further comprising drying the article.

68. The method of claim 67, wherein the article is dried at room temperature, freeze-dried, or dried in a heated oven.
